SEQUENCE LISTING (2 sequences)

SEQ ID NO: 1 - protein, 383 aa, Homo sapiens
  source 1..383: note = DLK1
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNRTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEGQAICFTILGVLTSLVVLGTVGIVFLNKCETWVSNLRYNHMLRKKKNLLLQYNSGEDLAVNIIFPEKIDMTTFSKEAGDEEI

SEQ ID NO: 2 - protein, 304 aa, Homo sapiens
  source 1..304: note = extracellular domain of DLK1
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNRTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEGQA

(19) United States
(12) Patent Application Publication
(10) Pub. No.: US 2024/0092857 A1
(43) Pub. Date: Mar. 21, 2024

(54) DELTA-LIKE NON-CANONICAL NOTCH LIGAND 1 ACTIVITY MODULATORS AND USES THEREOF

(71) Applicant: Fred Hutchinson Cancer Center, Seattle, WA (US)

(72) Inventors: Irwin D. Bernstein, Seattle, WA (US); Suzanne Furuyama, Mountlake Terrace, WA (US); Kristen Johnson, Seattle, WA (US)

(73) Assignee: Fred Hutchinson Cancer Center, Seattle, WA (US)

(21) Appl. No.: 18/456,339

(22) Filed: Aug. 25, 2023

Related U.S. Application Data
(60) Provisional application No. 63/492,712, filed on Mar. 28, 2023.
(60) Provisional application No. 63/373,675, filed on Aug. 26, 2022.

(51) Int. Cl. C07K 14/705; C12N 5/09

(52) U.S. Cl. CPC C07K 14/705; C12N 5/0694; A61K 38/00

(57) ABSTRACT

Delta-like non-canonical Notch ligand 1 (DLK1) inhibitors are disclosed. The DLK1 inhibitors can be used to treat cancers such as myelodysplastic syndrome (MDS), and cancers of the liver, breast, brain, pancreas, colon, lung, kidney, ovary, testes, and/or adrenal gland, among other uses described herein.

### CROSS REFERENCE TO RELATED APPLICATION

This application claims priority to U.S. Provisional Patent Application No. 63/373,675, filed on Aug. 26, 2022 and U.S. Provisional Patent Application No. 63/492,712 filed on Mar. 28, 2023, the contents of both of which are incorporated herein by reference in their entirety.

### STATEMENT REGARDING FEDERALLY SPONSORED RESEARCH OR DEVELOPMENT

This invention was made with government support under grant DK110563 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 2YB2776-Sequence Listing.xml. The file is 3,810 bytes, was created on Aug. 24, 2023, and is being submitted electronically via Patent Center.

### FIELD OF THE DISCLOSURE

The current disclosure provides delta-like non-canonical Notch ligand 1 (DLK1) activity modulators and uses thereof. Certain examples can be used to treat a variety of cancers or symptoms thereof including myelodysplastic syndrome (MDS), as well as cancers of the liver, breast, brain, pancreas, colon, lung, kidney, ovary, testes, and/or adrenal gland. The activity modulators can also be used to promote hematopoietic stem cell differentiation towards a myeloid lineage and induce precursor cycling allowing increased susceptibility to chemotherapy.

### BACKGROUND OF THE DISCLOSURE

Myelodysplastic syndrome (MDS) refers to a group of cancerous disorders in which blood cells in the bone marrow become abnormal or damaged due to a disorder of the hematopoietic stem cells. The hematopoietic stem cells develop into cancer cells, which results in the bone marrow making fewer red blood cells, white blood cells, and/or platelets. Because of this, and beyond having cancer, those with MDS are prone to low blood counts, anemia, infections, and increased likelihood of bleeding.

### SUMMARY OF THE DISCLOSURE

The current disclosure provides delta-like non-canonical Notch ligand 1 (DLK1) activity modulators and uses thereof.

DLK1 is 383 amino acids in length. The extracellular domain of DLK1 (ECD) spans from residue numbers 1-304 of the DLK1 sequence. This sequence is available under UniProt accession number P80370, NIH gene ID 8788. The extracellular domain of DLK1 ECD contains modular interacting domains (domains that interact with other proteins to modulate signaling), low complexity sequence domains (domains that are composed of similar amino acids resulting in low complexity within that domain) and a combination of ordered and intrinsically disordered regions that promote homo- and hetero-interactions to modulate cellular signaling.

In particular embodiments, DLK1 activity modulators modify a disease-linked mutation in the ECD, such as Arg160, Gly199, Arg267, and/or Val272.

In particular embodiments, DLK1 activity modulators modify a low complexity region of the ECD. For example pi-pi interacting residues 171, 173, 174, 186, 187, can be mutated (e.g., substituted and/or partially or completed deleted).

In particular embodiments, DLK1 activity modulators have a modified signal peptide (residues 1-23) through mutation (e.g., substitution and/or partial or complete deletion) of residues within the signal peptide.

In particular embodiments, DLK1 activity modulators have a mutation (e.g., insertion, substitution, or deletion), of any single or combination of the 36 cysteine residues within the ECD. Cysteine residues outside of the ECD may also be mutated.

In particular embodiments, DLK1 activity modulators have a combination of the modifications described above (disease linked mutation, modular interacting domains, low complexity regions, signal peptide, and/or cysteine residues) to modulate signaling.

DLK1 activity modulators can be used for a variety of purposes including in the treatment of a variety of cancers or symptoms thereof including myelodysplastic syndrome (MDS), as well as cancers of the liver, breast, brain, pancreas, colon, lung, kidney, ovary, testes, and/or adrenal gland. In particular embodiments, treatment of a cancer reduces blast counts, reduces cytopenia, and/or reduces neutropenia. In particular embodiments, DLK1 activity modulators induce precursor cycling allowing susceptibility to chemotherapy. Other potential uses are described elsewhere herein.

### BRIEF DESCRIPTION OF THE SEVERAL VIEWS OF THE DRAWINGS

Some of the drawings submitted herein may be better understood in color. The patent or application file contains at least one drawing executed in color. Copies of this patent or patent application publication with color drawing(s) will be provided by the Office upon request and payment of the necessary fee.

FIGS. 1A-1H. (FIG. 1A) UMAP and cluster analysis of combined DLK1 knockdown (KD) and Scrambled control (SCR) scRNAseq datasets derived from isolated human CB HSC ‘annotated by cell type based on canonical gene expression profiles. MPP=multipotent progenitors, MEP=Megakaryocyte/erythroid progenitors, GMP=granulocyte/monocyte progenitors. (FIG. 1B) UMAP showing proliferation index (a transcriptional measure of cell cycle activity). (FIG. 1C) Heatmap of gene set scores by cell type cluster for published gene sets associated with quiescent HSC state, activated, or cycling HSC/MPP state, proliferation index, or GMP. (FIG. 1D) UMAP by sample. (FIG. 1E) DLK1 expression by sample. (FIG. 1F) Proportion of cells by cell type from SCR vs DLK1 KD samples. (FIG. 1G) DLK1 transcript expression by cell type. (FIG. 1H) Gene-set scores for genes associated with quiescent HSC state, activated HSC/MPP state, or immature myeloid bias between cells from SCR vs DLK1 KD samples (limited to cells of qHSC and aHSC/MPP cell type clusters).

FIGS. 2A, 2B. Effect of DLK1 on HSPC: HSPC: DLK1 knockdown reduces in vivo repopulation, whereas DLK1 overexpression delays in vivo CD34+ cell differentiation.

FIG. 3. DLK1 knockdown leads to more rapid myeloid differentiation of HSPC during in vitro EC co-culture.

FIG. 4. DLK1 overexpression or exposure to exogenous DLK1 ECD leads to an increase in the number of CD34+CD90lo HSPC in vitro. CB CD34+ cells were transduced with lentiviruses encoding DLK1-GFP or Control GFP and cultured on Akt activated ECs for 2 weeks. Immunophenotype of GFP+ CD34+ CD90lo cells was determined by flow cytometry. P-value were determined using a t-test. Each of seven experiments is represented by a different shape.

FIG. 5. Exogenously presented ECD is capable of delaying differentiation of highly enriched CB derived HSC during ex vivo culture. CB CD34+Lin-CD38-CD90loCD45RA-EPCR+ cells were cultured with or without exogeneous ECD (10 ug/mL). On day 9, the proportion of CD34+ (left) and CD34+ CD90lo cells (right) was determined by flow cytometry. P values were determined using a t-test.

FIGS. 6A, 6B. DLK1 knockdown reduced TMRMlo CD34+ cells during EC co-culture. CB CD34+ cells were transduced with lentiviruses encoding DLK1 or SCR control shRNAs and co-expressing GFP and were cultured on Akt-activated ECs for 2 weeks. Immunophenotype of GFP+ cells was determined by flow cytometry. (FIG. 6A) Representative dot plots of CD34+ vs. TMRM staining in GFP+ SCR or DLK1 KD cells. (FIG. 6B) Bar graph showing the mean percent GFP+ CD34+ TMRMlo among three replicates. Error represents SEM. P-value were determined using a t-test. TMRM measures mitochondrial membrane potential.

FIG. 7. DLK1 expression in transcripts per million (TPM) among CD34⁺ cells from MDS patients (left) or normal bone marrows (right).

FIGS. 8A-8C. (8A) UMAP and cluster analysis of combined DLK1 knockdown and scrambled control datasets for MDS1. (8B) Cluster annotation using expression of lineage specific markers. (8C) The proportion of cells within each cluster for DLK1 knockdown (DLK_KD) and Scrambled control (SCR) datasets. Enriched in DLK1 KD (cluster 7, 8, 9); enriched in scrambled control (cluster 1, 2, 4).

FIGS. 9A-9C. Heatmap of gene set scores for signature gene sets defining (9A) HSC/MPP, (9B) immature myeloid precursors, or (9C) granulocytes in the DLK1 knockdown (DLK KD) or Scrambled control (SCR) datasets from MDS1.

FIG. 10. UMAP heatmap of DLK1 gene expression in the combined DLK1 knockdown and Scrambled control datasets from MDS1. Arrows depict the direction of lineage differentiation based on the annotations of clusters presented in FIGS. 8A-8C.

FIG. 11. DLK1 reduction enhances the ex vivo generation of granulocytes from MDS precursor cells in longer-term culture. CD34⁺ MDS precursors were isolated and infected with lentivirus expressing a shRNA specific for DLK1 or its scrambled control and co-expressing GFP. GFP⁺ transduced cells were cultured for three weeks on Akt-activated endothelial cells and a portion of the cells were analyzed for immune phenotype by flow cytometry. CD45 was used to separate MDS cells from ECs and GFP was used to select for transduced cells. CD34⁺CD15⁻ represent multipotent progenitors, CD34⁺CD15⁺ represent myeloid biased progenitors, CD34⁻ CD15⁺ represent granulocytes. DLK (left bars of pairs, marked with a chevron); scrambled (right bar of pairs, marked with a triangle).

FIGS. 12A-12D. Quiescence exit gene set score (FIGS. 12A, 12B) and cell cycle primed gene set score (FIGS. 12C, 12D) for the most primitive cluster identified in two independent MDS specimens.

FIG. 13. Membrane-associated and/or receptor complex component found to interact with DLK1. Eight membrane-associated and/or receptor complex components identified as statistically significant in IP-MS data. Significance was determined by a Median Fold change of greater than 2 as well as a False Discovery Rate (FDR)<0.01 in cells expressing DLK1-GFP compared to GFP control in three independent experiments.

FIGS. 14A, 14B. DLK1-ECD inhibits TGFβ signaling and Notch Receptor Cleavage. (FIG. 14A) KG1a-GFP cells were incubated in with (star) or without (triangle) 2 ng/mL TGFβ, in the presence (dashed line) or absence (solid line) of 10 ug/mL ECD. After 1 h incubation, cells were fixed, permeabilized, stained with anti-pSMAD2/3 antibody, and analyzed with flow cytometry. Flow plots from one representative experiment are shown (left panel) and % pSMAD2/3 positive cells are shown from replicates over 4 independent experiments. P value determined using a student's t-test. (FIG. 14B) CHO cells were incubated with immobilized DeltaextlgG in the presence of increasing concentrations of ECD (0, 1, 3 and 10 ug/mL) or the gamma-secretase inhibitor DAPT at 10 ug/mL. Cells were analyzed with western blot using an antibody against Notch1 receptor cleaved by gamma secretase at Valine 1744 (clone D3B8). Signal was normalized to actin per sample. Graph represents the average of 9 samples, analyzed in 3 independent experiments, error bars are SEM.

FIGS. 15A, 15B. Endogenously expressed DLK1 is capable of inhibiting pSMAD2 in response to TGFb ligand. (FIG. 15A) KG1a cells were plated with or without 1 ng/mL TGFb for 1 hr at 37° C. Cells were then fixed, permeabilized, and stained with anti-pSMAD2/3 antibody or its isotype control and analyzed by flow cytometry. Histograms marked with a star represent anti-pSMAD2/3 staining. Histograms marked with a triangle represents isotype control. Experiments were done in triplicate. (FIG. 15B) KG1a cells expressing full-length DLK1-GFP or GFP control (160,000 cells/well) were plated with or without TGFb (1 ng/mL) for 1 hr at 37° C. Cells were then fixed, permeabilized and stained with anti-pSMAD2/3 antibody or its isotype control and analyzed by flow cytometry. Histograms marked with a triangle represents anti-pSMAD2/3 staining in KG1a DLK1-GFP cells. Histograms marked with a star represent anti-pSMAD2/3 staining in KG1a GFP cells. Reactions were done in triplicate.

FIG. 16. DLK1 localization in the plasma membrane using the hematopoietic cell line KG1a as a model.

FIGS. 17A, 17B. Stimulated emission depletion (STED) microscopy revealed that plasma membrane localized DLK1 is not uniform but organized into particles that resemble phase separated condensates. (FIG. 17A) KG1a-DLK1-GFP cells were fixed and stained for ECD (Red). Confocal images were acquired of the center of the cell using a Zeiss LSM 789. (FIG. 17B) KG1a-DLK1-GFP cells were fixed and stained for ECD (Red). Images were acquired of the surface PM of cells using STED microscopy on a Lecia SP8.

FIG. 18. FL DLK1 and ECD exhibit phase separation tendencies. Cells were imaged as described in FIG. 17B. Ten images were analyzed per condition to determine the number of FL DLK1 condensates (left panel) and ECD condensates (right panel) per cell in control and 1,6-HD treated samples. P values were determined using a two tailed students t test.

FIGS. 19A-19F. DLK1 co-localizes with TGFBR1 and PSEN1. (FIG. 19A) KG1a-DLK1-GFP cells were seeded on a poly-lysine and fibronectin coated cover glass then fixed and stained with anti DLK1-ECD (594) and anti TGFBR1 (Atto-647N) antibodies. Images of the surface of the cell (PM) were acquired. Images were analyzed for ECD, GFP, and TGFBR1 condensates using FIJI. (FIG. 19B) The area of each DLK1 condensate −/+ TGFBR1 shown in nm². (FIG. 19C) ECD intensity per condensate −/+ TGFBR1 is shown in arbitrary units (AU). (FIG. 19D) Cells were prepared for microscopy as in 22A and stained with anti DLK1-ECD (594) and anti PSEN1(Atto-647N) antibodies. (FIG. 19E) The area of each DLK1 condensate −/+ PSEN1 shown in nm². (FIG. 19F) ECD intensity per condensate −/+ PSEN1 is shown in AU. P values were determined using a two tailed students t-test. Scale bars are 5 μm.

FIGS. 20A-20C. DLK1 alters receptor dynamics (FIG. 20A). KG1a cells were pretreated with 0, 1, or 10 ug/mL ECD then stimulated with 2 ng/mL TGFβ before fixing and staining for TGFBR1 and pSmad2/3 levels. Error bars are SEM. (FIG. 20B). KG1a-DLK1-GFP cells were fixed and stained for PSEN1(584) and either Rab7-647 (late endosomes) or Lamp1-647 (lysosome). Scale bars are 10 μm. (FIG. 20C) DLK1 co-localizes with both TGFBR1 and PSEN1 by microscopy. KG1a expressing full-length DLK1 fused to GFP were fixed and stained with anti-PSEN1 or anti-TGFBR2 antibodies. Cells were imaged using the 100× objective on a Zeiss LSM with Airyscan detection. Green=DLK1-GFP, Red=TGFBR2 or PSEN1.

FIG. 21. Schematics depicting DLK1 ECD mutants. Pr=ADAM17/TNFα converting enzyme (TACE) cleavage region; IgK=exogenous signal peptide; SP=endogenous DLK1 signal peptide; and HIS=polyhistidine tag. From N-terminal to C-terminal, epidermal growth factor like (EGF) domains can be referred to as EGF1, EGF2, EGF3, EGF4, EGFS, and EGF6.

### DETAILED DESCRIPTION

The Notch ligand family member, Delta like homologue 1 (DLK1), is essential to the development and homeostasis of tissues within each of the three germ layers. While broadly utilized during embryogenesis, DLK1 is restricted in usage to distinct lineages within the adult (for review, see (Traustadottir et al., Cytokine Growth Factor Rev. 2019; 46:17-27)). DLK1 is a master regulator of normal and malignant hematologic stem cells (HSC) and its expression is widely associated with aggressive and chemotherapeutically resistant cancers (Grassi et al., J Histochem Cytochem. 2022; 70(1):17-28), including in cancers of the liver, breast, brain, pancreas, colon, lung, kidney, ovary, testes, adrenal gland, and myelodysplastic syndrome (MDS) (Pittaway et al., Endocr Relat Cancer. 2021; 28(12):R271-R87).

The DLK1 gene encodes a single pass transmembrane protein composed of an extracellular domain (ECD) with an N-terminal signal peptide and six epidermal growth factor (EGF)-like domains; a juxtamembrane region containing an ADAM17/TNFα converting enzyme (TACE)-dependent proteolytic cleavage site; and a short cytoplasmic tail. Upon its insertion into the plasma membrane, DLK1 can remain membrane-bound or be proteolytically cleaved by TACE to generate a soluble ECD fragment. DLK1 is also regulated at the level of pre-mRNA splicing; in humans, the two predominant spliced isoforms differ in their incorporation of the TACE cleavage site (Traustadottir et al., Cytokine Growth Factor Rev. 2019; 46:17-27). The usage of the membrane-bound versus proteolytically cleaved isoforms varies dependent on biological context. Importantly, the generation of a soluble DLK1 ECD fragment allows DLK1 to function in both an autocrine and paracrine manner, where it may amplify or suppress membrane-bound DLK1 function.

DLK1 is 383 amino acids in length. This sequence is available under UniProt accession number P80370, NIH gene ID 8788, and includes:

(SEQ ID NO: 1)
MTATEALLRVLLLLLAFGHSTYGAECFPACNPQNGFCEDDNVCRCQPGWQ
GPLCDQCVTSPGCLHGLCGEPGQCICTDGWDGELCDRDVRACSSAPCANN
RTCVSLDDGLYECSCAPGYSGKDCQKKDGPCVINGSPCQHGGTCVDDEGR
ASHASCLCPPGFSGNFCEIVANSCTPNPCENDGVCTDIGGDFRCRCPAGF
IDKTCSRPVTNCASSPCQNGGTCLQHTQVSYECLCKPEFTGLTCVKKRAL
SPQQVTRLPSGYGLAYRLTPGVHELPVQQPEHRILKVSMKELNKKTPLLT
EGQAICFTILGVLTSLVVLGTVGIVFLNKCETWVSNLRYNHMLRKKKNLL
LQYNSGEDLAVNIIFPEKIDMTTFSKEAGDEEI.

The extracellular domain of DLK1 (ECD) spans from residue numbers 1-304 of the DLK1 sequence and includes:

(SEQ ID NO: 2)
MTATEALLRVLLLLLAFGHSTYGAECFPACNPQNGFCEDDNVCRCQPGWQ
GPLCDQCVTSPGCLHGLCGEPGQCICTDGWDGELCDRDVRACSSAPCANN
RTCVSLDDGLYECSCAPGYSGKDCQKKDGPCVINGSPCQHGGTCVDDEGR
ASHASCLCPPGFSGNFCEIVANSCTPNPCENDGVCTDIGGDFRCRCPAGF
IDKTCSRPVTNCASSPCQNGGTCLQHTQVSYECLCKPEFTGLTCVKKRAL
SPQQVTRLPSGYGLAYRLTPGVHELPVQQPEHRILKVSMKELNKKTPLLT
EGQA.

The extracellular domain of DLK1 ECD contains modular interacting domains (domains that interact with other proteins to modulate signaling), low complexity sequence domains (domains that are composed of similar amino acids resulting in low complexity within that domain) and a combination of ordered and intrinsically disordered regions that promote homo- and hetero-interactions to modulate cellular signaling.

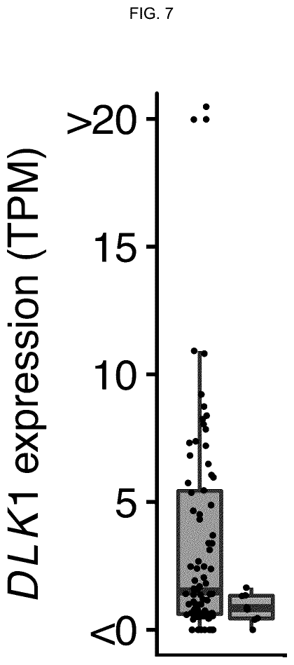

While the molecular basis is unknown (Benetatos et al., Leuk Res. 2010; 34(2):148-53, Anwar et al., PLoS One. 2012; 7(11):e49462; Greife et al., Clin Epigenetics. 2014; 6(1):29; Foong et al., Dev Cell. 2021; 56(22):3035-7), increased expression of DLK1 among CD34⁺ MDS precursors is associated with MDS progression (Sakajiri et al., Leukemia. 2005; 19(8):1404-10; Ma et al., Clin Lymphoma Myeloma Leuk. 2012; 12(4):261-8), including increased blast cell count and IPSS score (Yue et al., Cancer Biol Med. 2012; 9(3):188-91). To confirm these studies, RNAseq data from a cohort of 84 MDS specimens (Foong et al., Blood. 2018; 132(12):1225-40) was mined for DLK1 expression among CD34⁺ cells and increased levels relative to their normal counterparts (p=0.035) was found, with 35 patients exhibiting DLK1 expression above the median (FIG. 7). Taken together, the data show that increased DLK1 expression is a common, if not obligate, occurrence in advanced MDS.

Elucidating the mechanism(s) by which DLK1 functions to regulate stem cells has been complicated by the inability to clearly identify DLK1-interacting proteins. To date, studies have suggested that DLK1 promiscuously interacts with multiple receptor proteins including ACVR2B (Park et al., Activin Receptor Type II B Inhibitors Comprising DLK1 Extracellular Water-Soluble Domain. Google Patents; 2014), CFR (Miyaoka et al., Development. 2010; 137(1):159-67) and NOTCH1 (Baladron et al., Exp Cell Res. 2005; 303(2):343-59; Traustadottir et al., Cell Signal. 2016; 28(4):246-54), findings that in some cases are unsubstantiated (Park et al., Activin Receptor Type II B Inhibitors Comprising DLK1 Extracellular Water-Soluble Domain. Google Patents; 2014) and in others (e.g. Notch) inconsistent with structural data.

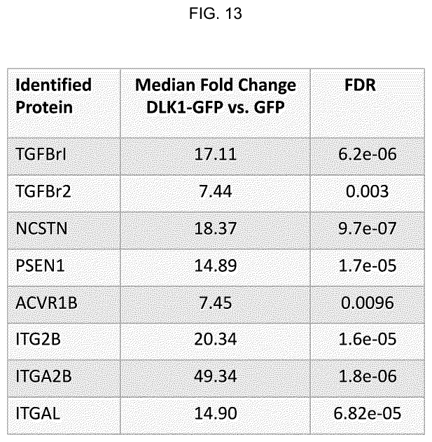

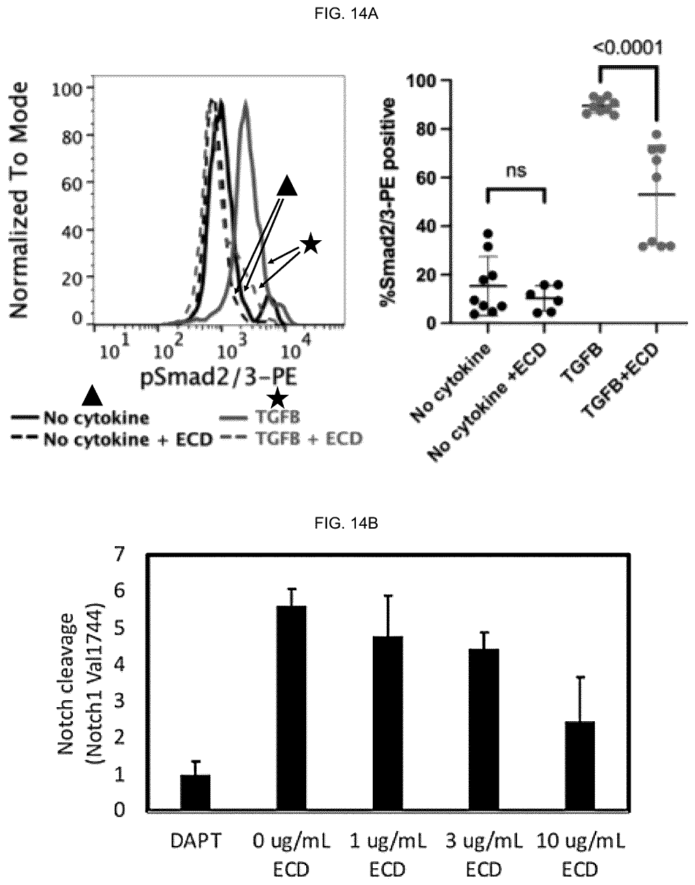

Using immunoprecipitation followed by mass spectrometry (IP-MS), reproducible enrichment of multiple proteins suggestive of promiscuous low affinity interactions, including members of the TGFb family of receptors as well as components of the g-secretase complex required for Notch activation was observed (FIG. 13). The implications of these latter interactions, were confirmed showing that DLK1 suppresses SMAD2 phosphorylation (pSMAD2) and reduces Notch receptor cleavage (FIGS. 14A, 14B).

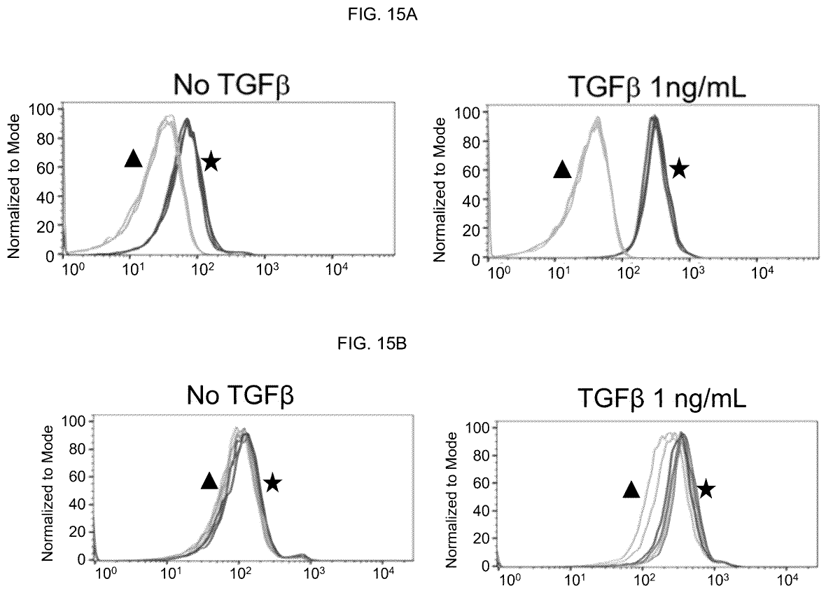

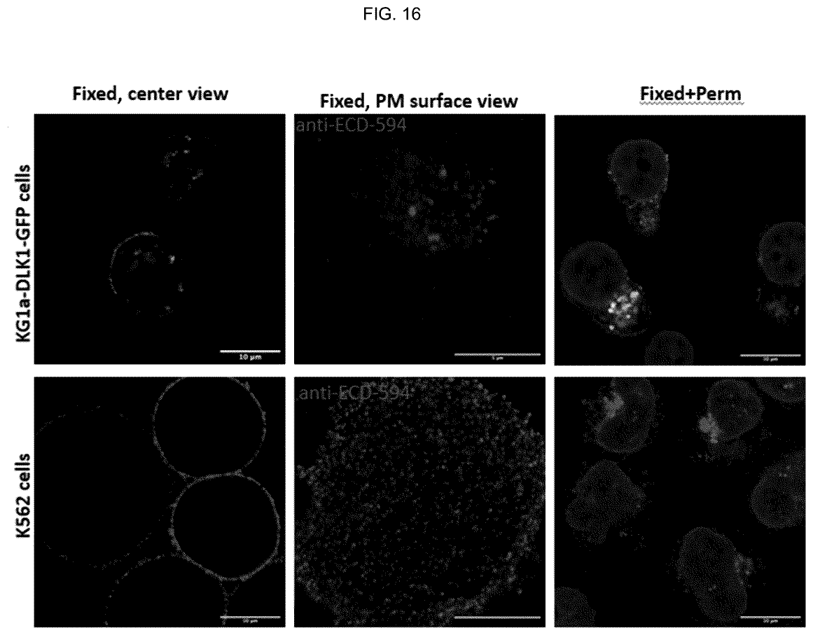

These promiscuous interactions may not be surprising as DLK1 is largely disordered. Rather than the conventional ‘lock and key’ structure-function paradigm, DLK1 may act like an intrinsically disordered protein (IDP; for review, see (Uversky et al., Frontiers in Physics. 2019; 7), engaging in low affinity interactions and condensate formation, the act of which has been shown to regulate receptor-mediated cell signaling (Case et al., Annu Rev Biophys. 2019; 48:465-94). In support of this, the distribution of DLK1 in the plasma membrane is not uniform, but rather dispersed in a cluster-like pattern resembling condensates (FIG. 16), data corroborated in (Huang et al., Carcinogenesis. 2007; 28(5):1094-103.). Also consistent with the notion that DLK1 functions as an IDP is the ability of DLK1 to self-associate (Baladron et al., Front Biosci. 2001; 6:A25-32), enabling the formation of a scaffold with enhanced avidity to strengthen otherwise weak protein-protein interactions (Ditlev et al., J Mol Biol. 2018; 430(23):4666-84). Thus, DLK1, by interfering with receptor interactions within condensates, can modulate signal transduction pathways essential to stem cell function both in normal and malignant hematopoiesis.

The current disclosure provides DLK1 activity modulators. In certain examples, the activity modulators are inhibitors that provide DLK1 modifications that reduce the natural function of DLK1. Particular embodiments modify one or more of: disease-linked mutations in the ECD, such as Arg160, Gly199, Arg267, and/or Val272; modular interaction domains of ECD; low complexity regions of ECD (e.g., pi-pi interacting residues 171, 173, 174, 186, 187); one or more signal peptide residues within residues 1-23; and/or one or more of the 36 cysteine residues within the ECD. Cysteine residues outside of the ECD may also be modified.

Particular embodiments mutate (e.g., substitute or delete) Arg160. Particular embodiments mutate (e.g., substitute or delete) Gly199. Particular embodiments mutate (e.g., substitute or delete) Arg267. Particular embodiments mutate (e.g., substitute or delete) Val272. Particular embodiments mutate (e.g., substitute or delete) Arg160 and Gly199. Particular embodiments mutate (e.g., substitute or delete) Arg160 and Arg267. Particular embodiments mutate (e.g., substitute or delete) Arg160 and Val272. Particular embodiments mutate (e.g., substitute or delete) Gly199 and Arg267. Particular embodiments mutate (e.g., substitute or delete) Gly199 and Val272. Particular embodiments mutate (e.g., substitute or delete) Arg267 and Val272. Antibodies against single or combination of disease linked mutations occurring in the ECD of DLK1 can also be used.

Antibodies targeting (e.g., binding) any single or combination of the modular interacting domains of DLK1-ECD to modulate ECD function can also be used.

Particular embodiments mutate (e.g., substitute or delete) Glu377. Particular embodiments mutate or delete Ala378. Particular embodiments mutate or delete Gly379. Particular embodiments mutate or delete Asp380. Particular embodiments mutate or delete Glu381. Particular embodiments mutate or delete Glu382. Particular embodiments mutate or delete lle383. Particular embodiments mutate or delete all of residues 378-383. Particular embodiments mutate or delete 2 of residues 378-383; 3 of residues 378-383; 4 of residues 378-383; or 5 of residues 378-383.

Particular embodiments mutate or delete Ala171. Particular embodiments mutate or delete residue Ser173. Particular embodiments mutate or delete residue Cys174. Particular embodiments mutate or delete residue Thr186. Particular embodiments mutate or delete residue Asp187. Particular embodiments mutate or delete all of residues Ala171, Ser173, Cys174, Thr186, and Asp187. Particular embodiments mutate or delete two of residues Ala171, Ser173, Cys174, Thr186, and Asp187; three of residues Ala171, Ser173, Cys174, Thr186, and Asp187; or four of residues Ala171, Ser173, Cys174, Thr186, and Asp187.

Antibodies targeting (e.g., binding) low complexity sequence regions within the ECD to modulate ECD function can also be used.

Certain examples expand the low complexity regions of ECD by insertion or site directed mutagenesis to generate new pi-pi interacting residues and low complexity sequences to promote homo-oligomerization with endogenous ECD and disrupt ECD-hetero interactions and modulate signaling.

Particular embodiments alter the ECD signal peptide, for example, mutation of and/or partial to complete deletion of residues within this region. Particular embodiments mutate and/or delete 1 residue of the signal peptide, 2 residues of the signal peptide, 3 residues of the signal peptide, 4 residues of the signal peptide, 5 residues of the signal peptide, 6 residues of the signal peptide, 7 residues of the signal peptide, 8 residues of the signal peptide, 9 residues of the signal peptide, 10 residues of the signal peptide, 11 residues of the signal peptide, 12 residues of the signal peptide, 13 residues of the signal peptide, 14 residues of the signal peptide, 15 residues of the signal peptide, 16 residues of the signal peptide, 17 residues of the signal peptide, 18 residues of the signal peptide, 19 residues of the signal peptide, 20 residues of the signal peptide, 21 residues of the signal peptide, 22 residues of the signal peptide, or 23 residues of the signal peptide.

Particular embodiments modify disulfide bonding and/or redox chemistry of the ECD molecule through mutation, insertion, and/or deletion of any single or combination of the 36 cysteine residues within the ECD and/or the two cysteine residues outside of the ECD. Particular embodiments mutate, insert, or delete 1 cysteine within the ECD, 2 cysteines within the ECD, 3 cysteines within the ECD, 4 cysteines within the ECD, 5 cysteines within the ECD, 6 cysteines within the ECD, 7 cysteines within the ECD, 8 cysteines within the ECD, 9 cysteines within the ECD, 10 cysteines within the ECD, 11 cysteines within the ECD, 12 cysteines within the ECD, 13 cysteines within the ECD, 14 cysteines within the ECD, 15 cysteines within the ECD, 16 cysteines within the ECD, 17 cysteines within the ECD, 18 cysteines within the ECD, 19 cysteines within the ECD, 20 cysteines within the ECD, 21 cysteines within the ECD, 22 cysteines within the ECD, 23 cysteines within the ECD, 24 cysteines within the ECD, 25 cysteines within the ECD, 26 cysteines within the ECD, 27 cysteines within the ECD, 28 cysteines within the ECD, 29 cysteines within the ECD, 30 cysteines within the ECD, 31 cysteines within the ECD, 32 cysteines within the ECD, 33 cysteines within the ECD, 34 cysteines within the ECD, 35 cysteines within the ECD, or 36 cysteines within the ECD. To mutate 37 or 38 cysteines, mutations will occur outside of the ECD. In certain examples, these embodiments utilize amino acid chemistry techniques to target engineered ECD-endogenous or ECD heterodimers for cellular degradation pathways such as with cysteine degraders (see ref: J. Am. Chem. Soc. 2020, 142, 11734-11742).

Cysteine residues within the ECD include Cys26, Cys30, Cys37, Cys43, Cys45, Cys54, Cys57, Cys63, Cys68, Cys74, Cys76, Cys85, Cys92, Cys97, Cys103, Cys113, Cys115, Cys124, Cys131, Cys138, Cys144, Cys156, Cys158, Cys167, Cys174, Cys179, Cys185, Cys194, Cys196, Cys205, Cys212, Cys217, Cys223, Cys233, Cys235, and Cys244.

Particular embodiments mutate, insert, or delete one or more of Cys26, Cys30, Cys37, Cys43, Cys45, Cys54, Cys57, Cys63, Cys68, Cys74, Cys76, Cys85, Cys92, Cys97, Cys103, Cys113, Cys115, Cys124, Cys131, Cys138, Cys144, Cys156, Cys158, Cys167, Cys174, Cys179, Cys185, Cys194, Cys196, Cys205, Cys212, Cys217, Cys223, Cys233, Cys235, and Cys244.

Particular embodiments mutate, insert, or delete one or more cysteine residues within EGF 5 and/or 6. Particular embodiments mutate, insert, or delete one or more Cys174, Cys179, Cys185, Cys194, Cys196, Cys205, Cys212, Cys217, Cys223, Cys233, Cys235, and Cys244.

Particular embodiments mutate, insert, or delete Cys174. Particular embodiments mutate, insert, or delete Cys179. Particular embodiments mutate, insert, or delete Cys185. Particular embodiments mutate, insert, or delete Cys194. Particular embodiments mutate, insert, or delete Cys196. Particular embodiments mutate, insert, or delete Cys205. Particular embodiments mutate, insert, or delete Cys212. Particular embodiments mutate, insert, or delete Cys217. Particular embodiments mutate, insert, or delete Cys223. Particular embodiments mutate, insert, or delete Cys235. Particular embodiments mutate, insert, or delete Cys233. Particular embodiments mutate, insert, or delete Cys244.

Particular embodiments mutate, insert, or delete two of Cys174, Cys179, Cys185, Cys194, Cys196, Cys205, Cys212, Cys217, Cys223, Cys233, Cys235, and Cys244. Particular embodiments mutate, insert, or delete three of Cys174, Cys179, Cys185, Cys194, Cys196, Cys205, Cys212, Cys217, Cys223, Cys233, Cys235, and Cys244. Particular embodiments mutate, insert, or delete four of Cys174, Cys179, Cys185, Cys194, Cys196, Cys205, Cys212, Cys217, Cys223, Cys233, Cys235, and Cys244. Particular embodiments mutate, insert, or delete five of Cys174, Cys179, Cys185, Cys194, Cys196, Cys205, Cys212, Cys217, Cys223, Cys233, Cys235, and Cys244. Particular embodiments mutate, insert, or delete six of Cys174, Cys179, Cys185, Cys194, Cys196, Cys205, Cys212, Cys217, Cys223, Cys233, Cys235, and Cys244. Particular embodiments mutate, insert, or delete seven of Cys174, Cys179, Cys185, Cys194, Cys196, Cys205, Cys212, Cys217, Cys223, Cys233, Cys235, and Cys244. Particular embodiments mutate, insert, or delete eight of Cys174, Cys179, Cys185, Cys194, Cys196, Cys205, Cys212, Cys217, Cys223, Cys233, Cys235, and Cys244. Particular embodiments mutate, insert, or delete nine of Cys174, Cys179, Cys185, Cys194, Cys196, Cys205, Cys212, Cys217, Cys223, Cys233, Cys235, and Cys244. Particular embodiments mutate, insert, or delete ten of Cys174, Cys179, Cys185, Cys194, Cys196, Cys205, Cys212, Cys217, Cys223, Cys233, Cys235, and Cys244. Particular embodiments mutate, insert, or delete eleven of Cys174, Cys179, Cys185, Cys194, Cys196, Cys205, Cys212, Cys217, Cys223, Cys233, Cys235, and Cys244. Particular embodiments mutate, insert, or delete all of Cys174, Cys179, Cys185, Cys194, Cys196, Cys205, Cys212, Cys217, Cys223, Cys233, Cys235, and Cys244.

In certain examples, the ECD of DLK1 inhibitors disclosed herein retain at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the ECD of a DLK1 reference sequence (e.g., SEQ ID NO: 2). In particular embodiments a modified domain of an DLK1 ECD retains at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the respective modified domain of a DLK1 reference sequence (e.g., UniProt accession number P80370, NIH gene ID 8788).

Antibodies that target (e.g., bnid) the ECD cysteine residues can also be used to modulate ECD function. Antibodies can target 1 or more of Cys26, Cys30, Cys37, Cys43, Cys45, Cys54, Cys57, Cys63, Cys68, Cys74, Cys76, Cys85, Cys92, Cys97, Cys103, Cys113, Cys115, Cys124, Cys131, Cys138, Cys144, Cys156, Cys158, Cys167, Cys174, Cys179, Cys185, Cys194, Cys196, Cys205, Cys212, Cys217, Cys223, Cys233, and Cys244

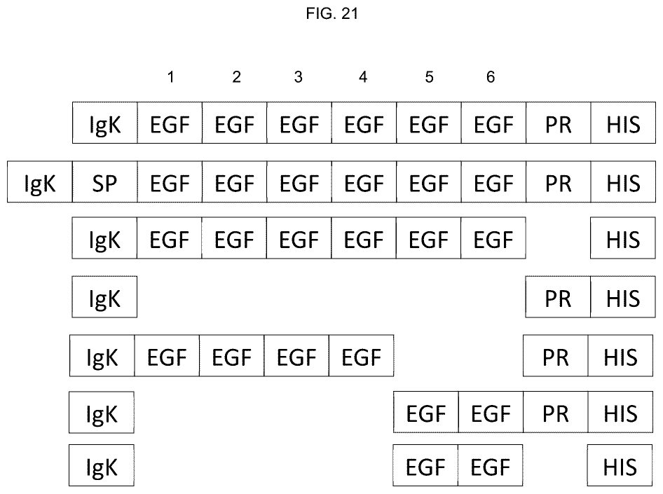

The current disclosure also provides single chain proteins having a modified DLK1 described herein. The single chain proteins can include, for example, an IgK signal peptide, a DLK1 signal peptide, 2 or more DLK1 EGF domains, an ADAM17/TNFα converting enzyme (TACE)-dependent proteolytic cleavage site, or a polyhistidine tag (His (e.g., 5, 6, 7, 8, 9, or 10 consecutive histidine residues). Schematics of particular embodiments of such single chain proteins are depicted in FIG. 21.

“Encoding” refers to the property of specific nucleotide sequences, such as cDNA or mRNA to serve as templates for synthesis of other macromolecules such as a defined sequence of amino acids. Thus, a nucleotide sequence encodes for a protein, for example, if transcription and translation of mRNA produces the protein in a cell or other biological system. A “nucleotide sequence encoding a protein” includes all nucleotide sequences that are degenerate versions of each other and that code for the same amino acid sequence.

Nucleotide sequences encoding proteins can be operably linked to relevant regulatory sequences. For example, there can be a functional linkage between a regulatory sequence and an exogenous nucleotide sequence resulting in expression of the latter. For another example, a first nucleotide sequence can be operably linked with a second nucleotide sequence when the first nucleotide sequence is placed in a functional relationship with the second nucleotide sequence. For instance, a promoter is operably linked to an encoding nucleotide sequence if the promoter affects the transcription or expression of the nucleotide sequence. Generally, operably linked DNA sequences are contiguous and, where necessary or helpful, join coding regions, into the same reading frame.

DLK1 inhibitors can be used to treat a variety of cancers or symptoms thereof including myelodysplastic syndrome (MDS), as well as cancers of the liver, breast, brain, pancreas, colon, lung, kidney, ovary, testes, and/or adrenal gland. In particular embodiments, treatment of a cancer reduces blast counts, reduces cytopenia, and/or reduces neutropenia.

Without being limited by theory, DLK1 may act as a master regulator of stem cells. Data presented herein indicates that DLK1 knockdown in malignant MDS precursors alters the balance of precursor proliferation and differentiation to enhance the generation of maturing granulocytes following short-term and long-term culture, indicating that DLK1 expression can cause disease progression by promoting MDS precursor self-renewal at the expense of differentiation. Stated more particularly, and again without being limited by theory, DLK1 knockdown can lead to the decreased self-renewal of primitive multipotent precursors and their differentiation towards the granulocytic lineage, as measured by increased expression of genes indicative of myeloid differentiation following short-term culture as well as cell surface myeloid markers indicative of granulocytic development following longer-term EC co-culture.

In certain examples, the current disclosure provides inhibition of DLK1 to promote hematopoietic stem cell differentiation towards a myeloid lineage. In particular embodiments, promoting hematopoietic stem cell differentiation towards a myeloid lineage reduces the expression of stemness markers. In particular embodiments, promoting hematopoietic stem cell differentiation towards a myeloid lineage includes driving HSC from a transcriptionally quiescent state to a more cycling state. In particular embodiments, promoting hematopoietic stem cell differentiation towards a myeloid lineage includes decreasing a proportion of multipotent progenitors within a cell sample and increasing the proportion of CD34+CD15+ granulocyte precursors and/or increasing the proportion of CD34−CD15+ granulocytes within the cell sample. In particular embodiments, promoting hematopoietic stem cell differentiation towards a myeloid lineage includes increasing immature myeloid and granulocytic gene set scores and decreasing HSC/MMP gene set scores within a cell sample.

Embodiments disclosed herein can also inhibit DLK1 to increase TGFb and/or Notch signaling.

Compositions for Administration. DLK1 inhibitors can be formulated into compositions for administration to subjects. Salts and/or pro-drugs of DLK1 inhibitors can also be used.

A pharmaceutically acceptable salt includes any salt that retains the activity of a DLK1 inhibitor and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or hydrolysis of a biologically labile group.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes DLK1 inhibitors or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the composition can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid compositions such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of the composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Depot compositions can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of nanoparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot compositions can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

In particular embodiments, the compositions include DLK1 inhibitors at at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers are disclosed in Remington's Pharmaceutical Sciences, 18th Ed. Mack Printing Company, 1990. Moreover, compositions and formulations can be prepared to meet sterility, pyrogenicity, general safety, and purity standards as required by U.S. FDA Office of Biological Standards and/or other relevant foreign regulatory agencies.

Methods of Use. Methods disclosed herein include treating subjects. Subjects include, e.g., humans, veterinary animals (dogs, cats, reptiles, birds) livestock (e.g., horses, cattle, goats, pigs, chickens) and research animals (e.g., monkeys, rats, mice, fish). Treating subjects includes delivering therapeutically effective amounts. Therapeutically effective amounts include those that provide effective amounts, prophylactic treatments and/or therapeutic treatments.

An “effective amount” is the amount of a composition or formulation necessary to result in a desired physiological change in the subject. Effective amounts are often administered for research purposes. Effective amounts disclosed herein can cause a statistically-significant effect in an animal model or in vitro assay relevant to the assessment of a condition's development, progression, and/or resolution.

A “prophylactic treatment” includes a treatment administered to a subject who does not display signs or symptoms of a condition or displays only early signs or symptoms of a condition such that treatment is administered for the purpose of diminishing or decreasing the risk of developing the condition further. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment, or therapeutic treatment are not mutually exclusive, and in particular embodiments, administered dosages may accomplish more than one treatment type.

In particular embodiments, therapeutically effective amounts provide anti-cancer effects. Anti-cancer effects can provide a decrease in the number of cancer cells, an increase in life expectancy, induced chemo- or radiosensitivity in cancer cells, inhibited cancer cell proliferation, prolonged subject life, reduced cancer-associated pain, and/or reduced relapse or re-occurrence of cancer following treatment.

Particular embodiments include treating myelodysplastic syndrome (MDS) and/or cancers of the liver, breast, brain, pancreas, colon, lung, kidney, ovary, testes, and/or adrenal gland.

In particular embodiments, therapeutically effective amounts reduce the expression of stemness markers in a population of hematopoietic stem cells.

In particular embodiments, therapeutically effective amounts drive a population of hematopoietic stem cells from a transcriptionally quiescent state to a more differentiated cell cycling state.

In particular embodiments, therapeutically effective amounts decrease a proportion of multipotent progenitors within a population of hematopoietic stem cells and increase the proportion of CD34+CD15+ granulocyte precursors and/or increase the proportion of CD34−CD15+ granulocytes within the population of hematopoietic stem cells.

In particular embodiments, therapeutically effective amounts increase immature myeloid and granulocytic gene set scores and decrease HSC/MMP gene set scores within a population of hematopoietic stem cells.

### Exemplary Embodiments

1. A modified DLK1 extracellular domain (ECD).
2. A modified DLK1 ECD of embodiment 1, wherein the modification includes a disease-linked mutation.
3. A modified DLK1 ECD of embodiment 2, wherein the disease-linked mutation is a mutation of Arg160, Gly199, Arg267, and/or Val272.
4. A modified DLK1 ECD of embodiment 3, wherein the disease-linked mutation includes a substitution or deletion of Arg160.
5. A modified DLK1 ECD of embodiment 3 or 4, wherein the disease-linked mutation includes a substitution or deletion of Gly199.
6. A modified DLK1 ECD of any of embodiments 3-5, wherein the disease-linked mutation includes a substitution or deletion of Arg267.
7. A modified DLK1 ECD of any of embodiments 3-6, wherein the disease-linked mutation includes a substitution or deletion of Val272.
8. A modified DLK1 ECD of any of embodiments 3-7, wherein the disease-linked mutation includes a substitution or deletion of Arg160 and Gly199.
9. A modified DLK1 ECD of any of embodiments 3-8, wherein the disease-linked mutation includes a substitution or deletion of Arg160 and Arg267.
10. A modified DLK1 ECD of any of embodiments 3-9, wherein the disease-linked mutation includes a substitution or deletion of Arg160 and Val272.
11. A modified DLK1 ECD of any of embodiments 3-10, wherein the disease-linked mutation includes a substitution or deletion of Gly199 and Arg267.
12. A modified DLK1 ECD of any of embodiments 3-11, wherein the disease-linked mutation includes a substitution or deletion of Gly199 and Val272.
13. A modified DLK1 ECD of any of embodiments 3-12, wherein the disease-linked mutation includes a substitution or deletion of Arg267 and Val272.
14. A modified DLK1 including a modified modular interacting domain.
15. A modified DLK1 protein of embodiment 14, wherein the modified modular interacting domain includes a substitution or deletion of Glu377.
16. A modified DLK1 protein of embodiment 14 or 15, wherein the modified modular interacting domain includes a substitution or deletion of Ala378.
17. The modified DLK1 protein of any of embodiments 14-16, wherein the modified modular interacting domain includes a substitution or deletion of Gly379.
18. The modified DLK1 protein of any of embodiments 14-17, wherein the modified modular interacting domain includes a substitution or deletion of Asp380.
19. The modified DLK1 protein of any of embodiments 14-18, wherein the modified modular interacting domain includes a substitution or deletion of Glu381.
20. The modified DLK1 protein of any of embodiments 14-19, wherein the modified modular interacting domain includes a substitution or deletion of Glu382.
21. The modified DLK1 protein of any of embodiments 14-20, wherein the modified modular interacting domain includes a substitution or deletion of lle383.
22. The modified DLK1 protein of any of embodiments 14-21, wherein the modified modular interacting domain includes a substitution or deletion of residues 377-383.
23. The modified DLK1 protein of any of embodiments 14-22, wherein the modified modular interacting domain includes a substitution or deletion of 2 of residues 377-383; 3 of residues 377-383; 4 of residues 377-383; or 5 of residues 377-383.
24. The modified DLK1 protein of any of embodiments 14-23, wherein the modified DLK1 retains at least 70%, 71%, 72%, 73%, 74%, 75%, 76%, 77%, 78%, 79%, 80%, 81%, 82%, 83%, 84%, 85%, 86%, 97%, 88%, 89%, 90%, 91%, 92%, 93%, 94%, 95%, 96%, 97%, 98%, or 99% sequence identity with the sequence as set forth as SEQ ID NO: 2.
25. The modified DLK1 ECD of any of embodiments 1-13, having a modified low complexity region.
26. The modified DLK1 ECD of embodiment 25, wherein the modified low complexity region includes a substitution or deletion of Ala171.
27. The modified DLK1 ECD of embodiment 25 or 26, wherein the modified low complexity region includes a substitution or deletion of Ser173.
28. The modified DLK1 ECD of any of embodiments 25-27, wherein the modified low complexity region includes a substitution or deletion of Cys174.
29. The modified DLK1 ECD of any of embodiments 25-28, wherein the modified low complexity region includes a substitution or deletion of Thr186.
30. The modified DLK1 ECD of any of embodiments 25-29, wherein the modified low complexity region includes a substitution or deletion of Asp187.
31. The modified DLK1 ECD of any of embodiments 25-30, wherein the modified low complexity region includes a substitution or deletion of Ala171, Ser173, Cys174, Thr186, and Asp187.
32. The modified DLK1 ECD of any of embodiments 25-31, wherein the modified low complexity region includes a substitution or deletion of two of residues Ala171, Ser173, Cys174, Thr186, and Asp187; three of residues Ala171, Ser173, Cys174, Thr186, and Asp187; or four of residues Ala171, Ser173, Cys174, Thr186, and Asp187.
33. The modified DLK1 ECD of any of embodiments 1-13 or 25-32, having a modified signal peptide.
34. The modified DLK1 ECD of embodiment 33, wherein the modified signal peptide includes a substitution or deletion of 1 residue of the signal peptide, 2 residues of the signal peptide, 3 residues of the signal peptide, 4 residues of the signal peptide, 5 residues of the signal peptide, 6 residues of the signal peptide, 7 residues of the signal peptide, 8 residues of the signal peptide, 9 residues of the signal peptide, 10 residues of the signal peptide, 11 residues of the signal peptide, 12 residues of the signal peptide, 13 residues of the signal peptide, 14 residues of the signal peptide, 15 residues of the signal peptide, 16 residues of the signal peptide, 17 residues of the signal peptide, 18 residues of the signal peptide, 19 residues of the signal peptide, 20 residues of the signal peptide, 21 residues of the signal peptide, 22 residues of the signal peptide, or 23 residues of the signal peptide.
35. The modified DLK1 ECD of any of embodiments 1-13 or 25-34, having at least one modified cysteine residue.
36. The modified DLK1 ECD of embodiment 35, wherein the modified cysteine residue is substituted, deleted, inserted, or modified with a cysteine degrader.
37. The modified DLK1 ECD of embodiment 35 or 36, wherein the modified cysteine is Cys174, Cys179, Cys185, Cys194, Cys196, Cys205, Cys212, Cys217, Cys223, Cys233, Cys235, or Cys244.
38. The modified DLK1 ECD of any of embodiments 35-37, wherein the modified cysteine is Cys174.
39. The modified DLK1 ECD of embodiment 38, further including a modified Cys179, Cys185, Cys194, Cys196, Cys205, Cys212, Cys217, Cys223, Cys233, Cys235, or Cys244.
40. The modified DLK1 ECD of any of embodiments 35-37, wherein the modified cysteine is Cys179.
41. The modified DLK1 ECD of embodiment 40, further including a modified Cys174, Cys185, Cys194, Cys196, Cys205, Cys212, Cys217, Cys223, Cys233, Cys235, or Cys244.
42. The modified DLK1 ECD of any of embodiments 35-37, wherein the modified cysteine is Cys185.
43. The modified DLK1 ECD of embodiment 42, further including a modified Cys174, Cys179, Cys194, Cys196, Cys205, Cys212, Cys217, Cys223, Cys233, Cys235, or Cys244.
44. The modified DLK1 ECD of any of embodiments 35-37, wherein the modified cysteine is Cys194.
45. The modified DLK1 ECD of embodiment 44, further including a modified Cys174, Cys179, Cys185, Cys196, Cys205, Cys212, Cys217, Cys223, Cys233, Cys235, or Cys244.
46. The modified DLK1 ECD of any of embodiments 35-37, wherein the modified cysteine is Cys196.
47. The modified DLK1 ECD of embodiment 46, further including a modified Cys174, Cys179, Cys185, Cys194, Cys205, Cys212, Cys217, Cys223, Cys233, Cys235, or Cys244.
48. The modified DLK1 ECD of any of embodiments 35-37, wherein the modified cysteine is Cys205.
49. The modified DLK1 ECD of embodiment 48, further including a modified Cys174, Cys179, Cys185, Cys194, Cys196, Cys212, Cys217, Cys223, Cys233, Cys235, or Cys244.
50. The modified DLK1 ECD of any of embodiments 35-37, wherein the modified cysteine is Cys212.
51. The modified DLK1 ECD of embodiment 50, further including a modified Cys174, Cys179, Cys185, Cys194, Cys196, Cys205, Cys217, Cys223, Cys233, Cys235, or Cys244.
52. The modified DLK1 ECD of any of embodiments 35-37, wherein the modified cysteine is Cys217.
53. The modified DLK1 ECD of embodiment 52, further including a modified Cys174, Cys179, Cys185, Cys194, Cys196, Cys205, Cys212, Cys223, Cys233, Cys235, or Cys244.
54. The modified DLK1 ECD of any of embodiments 35-37, wherein the modified cysteine is Cys223.
55. The modified DLK1 ECD of embodiment 54, further including a modified Cys174, Cys179, Cys185, Cys194, Cys196, Cys205, Cys212, Cys217, Cys233, Cys235, or Cys244.
56. The modified DLK1 ECD of any of embodiments 35-37, wherein the modified cysteine is Cys233.
57. The modified DLK1 ECD of embodiment 56, further including a modified Cys174, Cys179, Cys185, Cys194, Cys196, Cys205, Cys212, Cys217, Cys233, Cys235, or Cys244.
58. The modified DLK1 ECD of any of embodiments 35-37, wherein the modified cysteine is Cys235.
59. The modified DLK1 ECD of embodiment 58, further including a modified Cys174, Cys179, Cys185, Cys194, Cys196, Cys205, Cys212, Cys217, Cys223, Cys233, or Cys244.
60. The modified DLK1 ECD of any of embodiments 35-37, wherein the modified cysteine is Cys244.
61. The modified DLK1 ECD of embodiment 60, further including a modified Cys174, Cys179, Cys185, Cys194, Cys196, Cys205, Cys212, Cys217, Cys223, Cys235, or Cys233.
62. The modified DLK1 ECD of any of embodiments 35-37, wherein the modified cysteine is Cys37, Cys63, Cys131, Cys205, or Cys234.
63. The modified DLK1 ECD of any of embodiments 1-13 or 25-62, having 2-36 modified cysteine residues.
64. The modified DLK1 ECD of embodiment 63, wherein 2 cysteine residues, 3 cysteine residues, 4 cysteine residues, 5 cysteine residues, 6 cysteine residues, 7 cysteine residues, 8 cysteine residues, 9 cysteine residues, 10 cysteine residues, 11 cysteine residues, 12 cysteine residues, 13 cysteine residues, 4 cysteine residues, 15 cysteine residues, 16 cysteine residues, 17 cysteine residues, 18 cysteine residues, 19 cysteine residues, 20 cysteine residues, 21 cysteine residues, 22 cysteine residues, 23 cysteine residues, 24 cysteine residues, 25 cysteine residues, 26 cysteine residues, 27 cysteine residues, 28 cysteine residues, 29 cysteine residues, 30 cysteine residues, 31 cysteine residues, 32 cysteine residues, 33 cysteine residues, 34 cysteine residues, 35 cysteine residues, or 36 cysteine residues are substituted, deleted, inserted, or modified with a cysteine degrader.
65. The modified DLK1 ECD of embodiment 64, having at least two modified cysteine residues, wherein at least one of the modified cysteine residues is Cys174, Cys179, Cys185, Cys194, Cys196, Cys205, Cys212, Cys217, Cys223, Cys233, Cys235, or Cys244.
66. The modified DLK1 ECD of embodiment 64 or 65, having at least two modified cysteine residues, wherein at least one of the modified cysteine residues is Cys174.
67. The modified DLK1 ECD of any of embodiments 1-13 or 25-66, wherein the modified DLK1 ECD retains at least 70%, 71%, 72%, 73%, 74%, 75%, 76%, 77%, 78%, 79%, 80%, 81%, 82%, 83%, 84%, 85%, 86%, 97%, 88%, 89%, 90%, 91%, 92%, 93%, 94%, 95%, 96%, 97%, 98%, or 99% sequence identity with the sequence as set forth as SEQ ID NO: 2.
68. The modified DLK1 ECD of any of embodiments 1-13 or 25-67 as part of a full length DLK1 protein.
69. A single chain protein including a modified DLK1 ECD of any of embodiments 1-13 or 25-66 or a modified DLK1 protein of any of embodiments 14-24.
70. The single chain protein of embodiment 69, further including a signal peptide.
71. The single chain protein of embodiment 70, wherein the signal peptide includes an IgK signal peptide or a DLK1 signal peptide.
72. The single chain protein of embodiments 69 or 70, further including an IgK signal peptide and a DLK1 signal peptide.
73. The single chain protein of any of embodiments 69-72, wherein the modified DLK1 ECD includes at least two EGF domains.
74. The single chain protein of any of embodiments 69-73, wherein the modified DLK1 ECD includes 3, 4, 5, or 6 EGF domains.
75. The single chain protein of any of embodiments 69-73, wherein the modified DLK1 ECD includes EGF5 and EGF6.
76. The single chain protein of any of embodiments 69-75, wherein the modified DLK1 ECD includes EGF1, EGF2, EGF3, and EGF4.
77. The single chain protein of any of embodiments 69-76, wherein the modified DLK1 ECD includes EGF1, EGF2, EGF3, EGF4, EGF5, and EGF6.
78. The single chain protein of any of embodiments 69-77, further including an ADAM17/TNFα converting enzyme (TACE)-dependent proteolytic cleavage site.
79. The single chain protein of any of embodiments 69-78, further including a polyhistidine tag.
80. A nucleotide sequence encoding a modified DLK1 ECD of any of embodiments 1-13 or 25-68, a modified DLK protein of any of embodiments 14-25, or single chain protein of any of embodiments 69-79.
81. A nucleotide sequence of embodiment 80, wherein the nucleotide sequence is DNA or RNA.
82. A nucleotide sequence of embodiment 81, wherein the RNA is messenger RNA (mRNA) or in vitro synthesized RNA.
83. A composition including (i) a modified DLK1 ECD, a modified DLK protein, a single chain protein, or a nucleotide sequence of any of the preceding embodiments and (ii) a pharmaceutically acceptable carrier.
84. A method of treating cancer in a subject in need thereof including administering to the subject a therapeutically effective amount of the composition of embodiment 83, thereby treating the cancer in the subject in need thereof.
85. The method of embodiment 84, wherein the cancer is myelodysplastic syndrome (MDS).
86. The method of embodiment 85, wherein the MDS includes multilineage dysplasia.
87. The method of embodiment 84, wherein the cancer is cancer of the liver, breast, brain, pancreas, colon, lung, kidney, ovary, testes, and/or adrenal gland.
88. A method of promoting hematopoietic stem cell differentiation towards a myeloid lineage including administering a therapeutically effective amount of a DLK1 inhibitor disclosed herein to a population of hematopoietic stem cells, thereby promoting hematopoietic stem cell differentiation towards a myeloid lineage.
89. The method of embodiment 88, wherein the promoting the hematopoietic stem cell differentiation towards a myeloid lineage reduces the expression of stemness markers within the population of hematopoietic stem cells.
90. The method of embodiment 88 or 89, wherein the promoting the hematopoietic stem cell differentiation towards a myeloid lineage drives HSC from a transcriptionally quiescent state to a more differentiated cell cycling state within the population of hematopoietic stem cells.
91. The method of any of embodiments 88-90, wherein the promoting the hematopoietic stem cell differentiation towards a myeloid lineage decreases a proportion of multipotent progenitors within the population of hematopoietic stem cells and increases the proportion of CD34+CD15+ granulocyte precursors and/or increases the proportion of CD34−CD15+granulocytes within the population of hematopoietic stem cells.
92. The method of any of embodiments 88-91, wherein the promoting the hematopoietic stem cell differentiation towards a myeloid lineage increases immature myeloid and granulocytic gene set scores and decreases HSC/MMP gene set scores within the population of hematopoietic stem cells.
93. The method of any of embodiments 88-92, wherein the population of hematopoietic stem cells is within a subject.
94. The method of any of embodiments 88-93, wherein the promoting the hematopoietic stem cell differentiation induces precursor cycling increasing susceptibility to chemotherapy.
95. The method of embodiment 93 or 94, wherein the subject has a cancer.
96. The method of any of embodiments 93-95, wherein the promoting hematopoietic stem cell differentiation towards a myeloid lineage reduces blast count within the subject.
97. The method of any of embodiments 93-96, wherein the promoting hematopoietic stem cell differentiation towards a myeloid lineage reduces cytopenia within the subject.
98. The method of any of embodiments 93-97, wherein the promoting hematopoietic stem cell differentiation towards a myeloid lineage reduces neutropenia within the subject.
99. The method of any of embodiments 95-98, wherein the cancer is cancer of the liver, breast, brain, pancreas, colon, lung, kidney, ovary, testes, and/or adrenal gland.
100. The method of any of embodiments 88-99, wherein the DLK1 inhibitor is a modified extracellular domain of DLK1.
101. The method of embodiment 100, wherein the extracellular domain of DLK1 includes six epidermal growth factor (EGF)-like domains.
102. The method of embodiment 100 or 101, wherein the extracellular domain of DLK1 includes an ADAM17/TNFα converting enzyme (TACE)-dependent proteolytic cleavage site.
103. A method of increasing TGFβ signaling and/or Notch signaling including administering a therapeutically effective amount of a DLK1 inhibitor to a population of hematopoietic stem cells, thereby increasing TGFβ signaling and/or Notch signaling.
104. The method of embodiment 103, wherein the increased TGFβ signaling and/or Notch signaling reduces the expression of stemness markers within the population of hematopoietic stem cells.
105. The method of embodiment 103 or 104, wherein the increased TGFβ signaling and/or Notch signaling drives HSC from a transcriptionally quiescent state to a more differentiated cell cycling state within the population of hematopoietic stem cells.
106. The method of any of embodiments 103-105, wherein the increased TGFβ signaling and/or Notch signaling decreases a proportion of multipotent progenitors within the population of hematopoietic stem cells and increases the proportion of CD34+CD15+ granulocyte precursors and/or increases the proportion of CD34−CD15+ granulocytes within the population of hematopoietic stem cells.
107. The method of any of embodiments 103-106, wherein the increased TGFβ signaling and/or Notch signaling increases immature myeloid and granulocytic gene set scores and decreases HSC/MMP gene set scores within the population of hematopoietic stem cells.
108. The method of any of embodiments 103-107, wherein the population of hematopoietic stem cells is within a subject.
109. The method of embodiment 108, wherein the subject has a cancer.
110. The method of embodiment 109, wherein the cancer is myelodysplastic syndrome (MDS).
111. The method of embodiment 110, wherein the MDS includes multilineage dysplasia.
112. The method of any of embodiments 108-111, wherein the increased TGFβ signaling and/or Notch signaling reduces blast count within the subject.
113. The method of any of embodiments 108-112, wherein the increased TGFβ signaling and/or Notch signaling reduces cytopenia within the subject.
114. The method of any of embodiments 108-113, wherein the increased TGFβ signaling and/or Notch signaling reduces neutropenia within the subject.
115. The method of embodiment 109, wherein the cancer is cancer of the liver, breast, brain, pancreas, colon, lung, kidney, ovary, testes, and/or adrenal gland.
116. The method of any of embodiments 103-111, wherein the DLK1 inhibitor is a modified extracellular domain of DLK1.
117. The method of embodiment 116, wherein the extracellular domain of DLK1 includes six epidermal growth factor (EGF)-like domains.
118. The method of embodiment 117, wherein the extracellular domain of DLK1 includes an ADAM 17/TNFα converting enzyme (TACE)-dependent proteolytic cleavage site.

Particular embodiments include disease linked mutations in ECD. In particular embodiments, disease linked mutations of all residues within the ECD of DLK1 including of residues Arg160, Gly199, Arg267, Val272, which are implicated in any disease expressing DLK1 including MDS and other DLK1 expressing cancers, may decrease or increase interaction to 95%, 96%, 97%, 98%, or 99% sequence similarity of the ECD molecule. Particular embodiments include partial to complete deletion of residues to decrease or increase interactions and modulate signaling in MDS and other cancers that expresses DLK1. Particular embodiments include antibodies against single or combination of disease linked mutations occurring in the ECD of DLK1.

Particular embodiments include modified modular interaction domains of ECD. In particular embodiments, modular interacting domains EGF and EGF like calcium binding domains within the ECD are targeted through mutation. In particular embodiments, the position of these modular interacting domains is modified. Particular embodiments include partial to complete deletion of residues within this region to 80%, 81%, 82%, 83%, 84%, 85%, 86%, 87%, 88%, 89%, 90%, 91%, 92%, 93%, 94%, 95%, 96%, 97%, 98%, or 99%, sequence similarity of the ECD molecule. Particular embodiments include partial to complete deletion of residues within this region to decrease or increase interactions and modulate signaling in MDS and other cancers that expresses DLK1. Particular embodiments include antibodies targeting (e.g., binding) any single or combination of the modular interacting domains of DLK1-ECD to modulate ECD function.

Particular embodiments include modified low complexity regions of DLK1. Particular embodiments include generating a molecule with a modified low complexity sequence regions within the DLK1 including acidic residues 377-383 and pi-pi interacting residues 171, 173, 174, 186, 187 through mutation and/or partial to complete deletion of residues within this region to 94%, 95%, 96%, 97%, 98%, or 99% to 94%, 95%, 96%, 97%, 98%, or 99%, sequence similarity of the DLK1 molecule. Particular embodiments include partial to complete deletion of residues within this region to decrease or increase interactions and modulate signaling in MDS and other cancers that expresses DLK1. Particular embodiments include antibodies targeting (e.g., binding) low complexity sequence regions within the DLK1 to modulate DLK1 function. Particular embodiments include expanding the low complexity regions of ECD. Particular embodiments include insertion or site directed mutagenesis to generate new pi-pi interacting residues and low complexity sequences to promote homo-oligomerization with endogenous ECD and disrupt ECD-hetero interactions and modulate signaling in MDS and other cancers that expresses DLK1.

Particular embodiments include a modified ECD signal peptide. Particular embodiments include generating a molecule with a modified signal peptide (residues 1-23) through mutation to 94%, 95%, 96%, 97%, 98%, or 99%, sequence similarity of the ECD molecule. Particular embodiments include partial to complete deletion of residues within this region to decrease or increase interactions and modulate signaling in MDS and other cancers that expresses DLK1. Particular embodiments include generating an antibody against the signal peptide of ECD to modulate ECD function.

Particular embodiments include modified cysteine residues in ECD. Particular embodiments include modification of disulfide bonding and/or redox chemistry of the ECD molecule through mutation, insertion, and/or deletion of any single or combination of the 36 cysteine residues within the ECD to 80%, 81%, 82%, 83%, 84%, 85%, 86%, 87%, 88%, 89%, 90%, 91%, 92%, 93%, 94%, 95%, 96%, 97%, 98%, or 99% sequence identity. Particular embodiments include utilizing amino acid chemistry techniques to target engineered ECD-endogenous or ECD heterodimers for cellular degradation pathways such as with cysteine degraders (see ref: J. Am. Chem. Soc. 2020, 142, 11734-11742). Particular embodiments include generating an antibody against the ECD cysteine residues to modulate ECD function. Particular embodiments include utilizing amino acid chemistry techniques to target ECD-antibody complexes for cellular degradation pathways, such as with cysteine degraders.

Particular embodiments include optimized ECD variant protein containing a combination of modified residues from the ECD features of interest (disease linked mutation, modular interacting domains, low complexity regions, signal peptide, cysteine residues) to 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to ECD to modulate signaling in MDS and other cancers that expresses DLK1.

Experimental Example. DLK1 knockdown leads to egress of HSC from a transcriptionally quiescent to a cycling primed/cycling state.

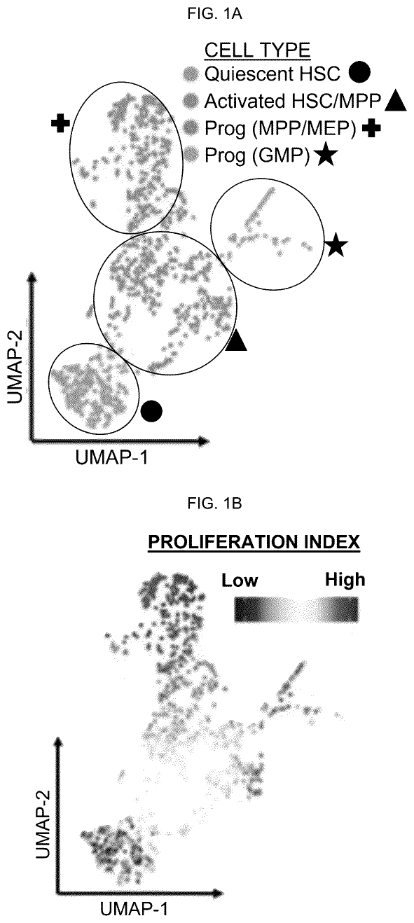

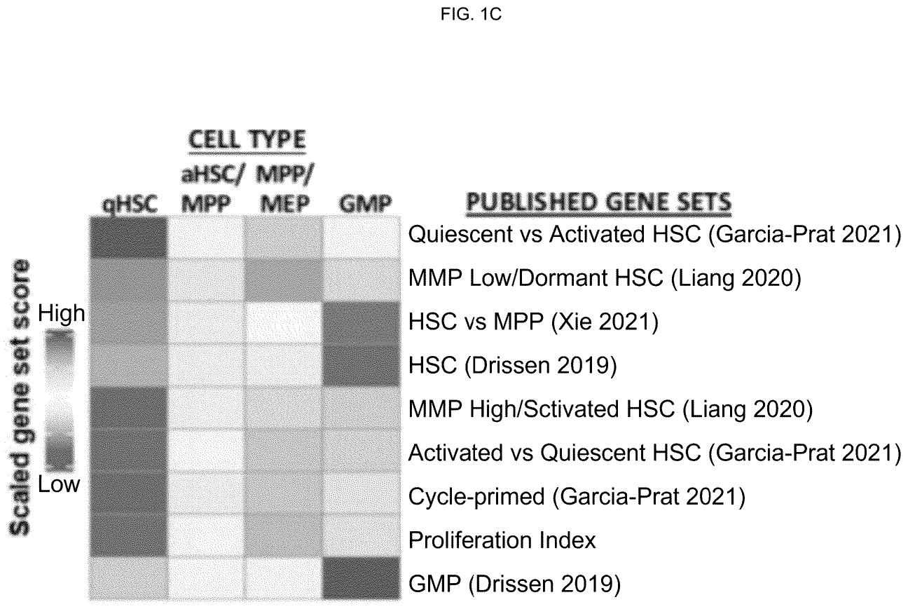

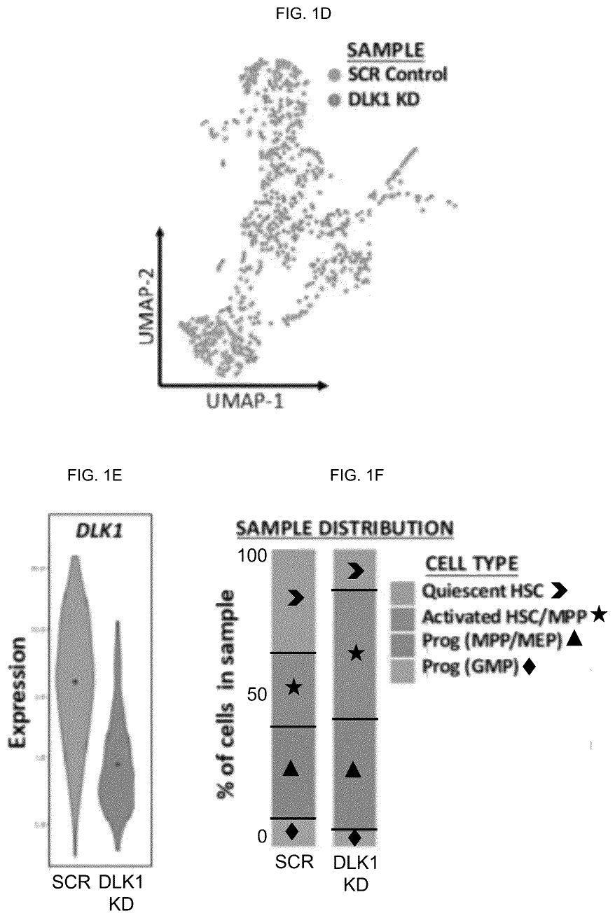

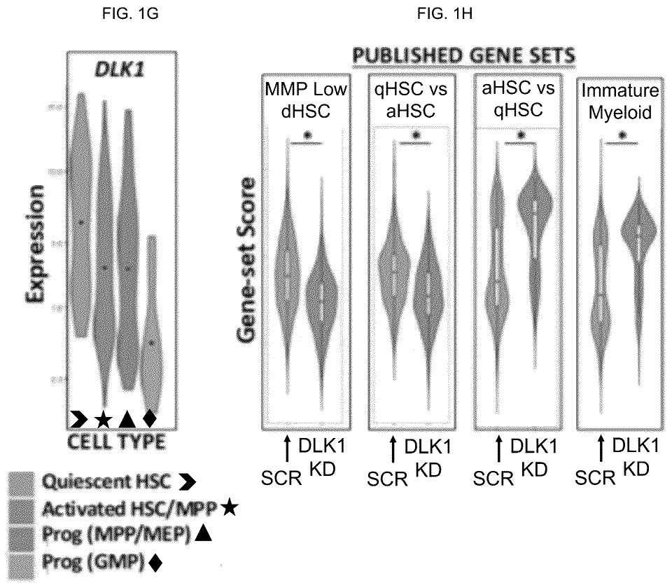

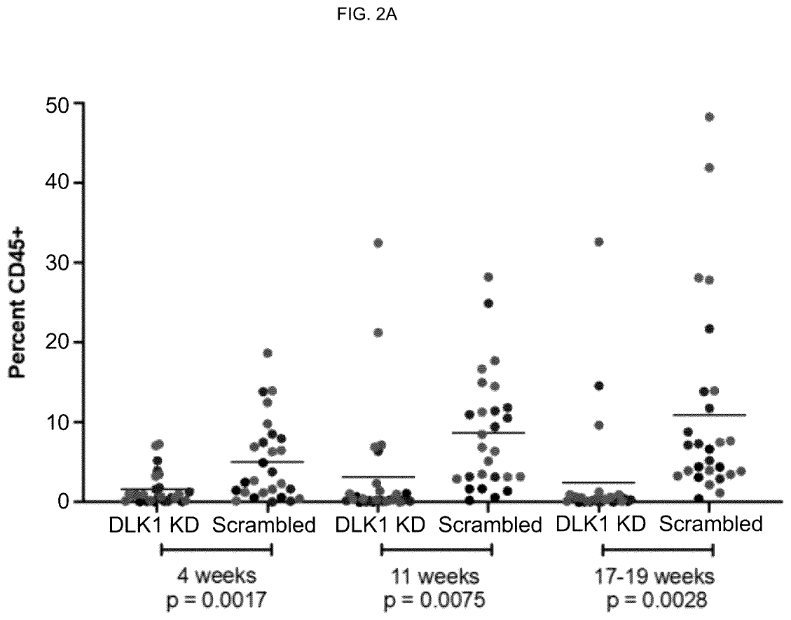

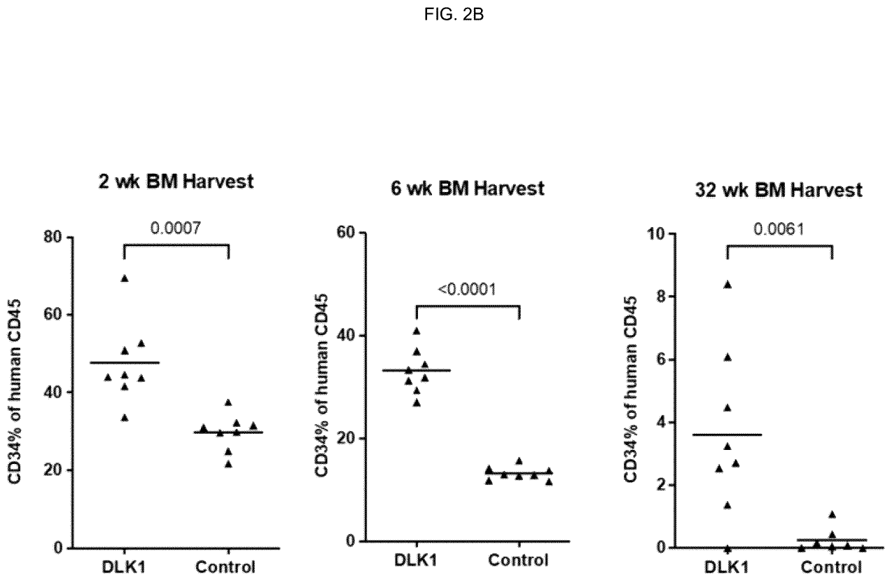

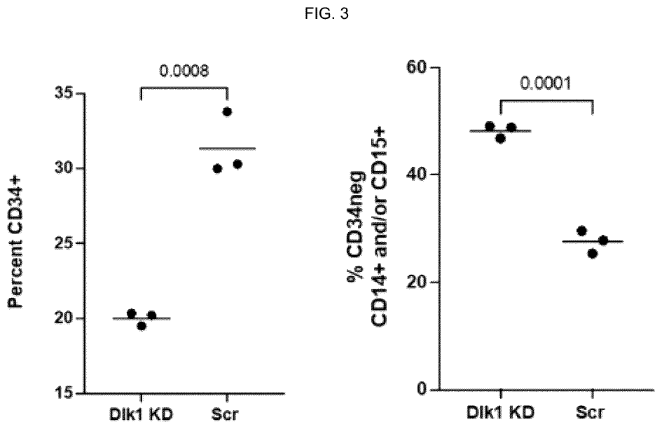

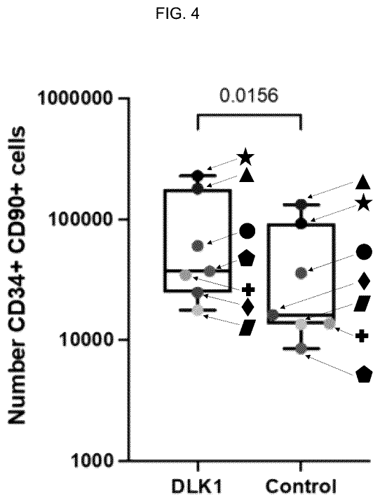

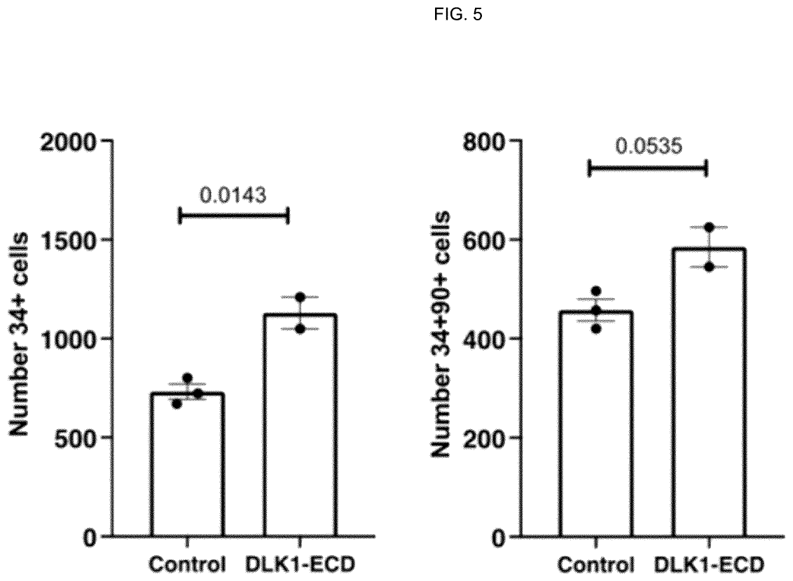

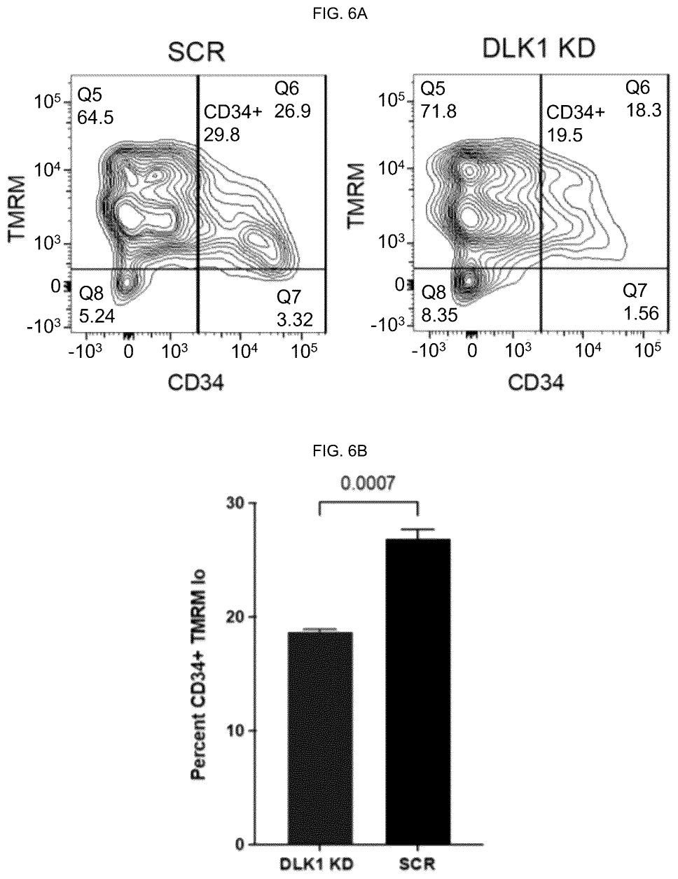

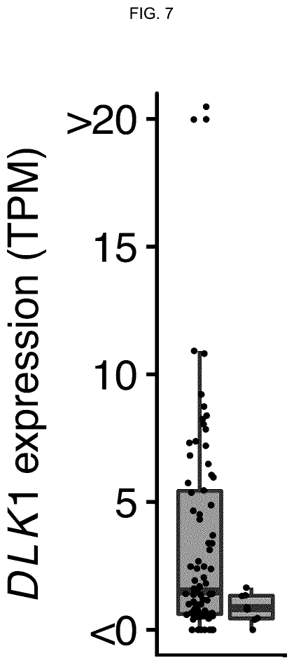

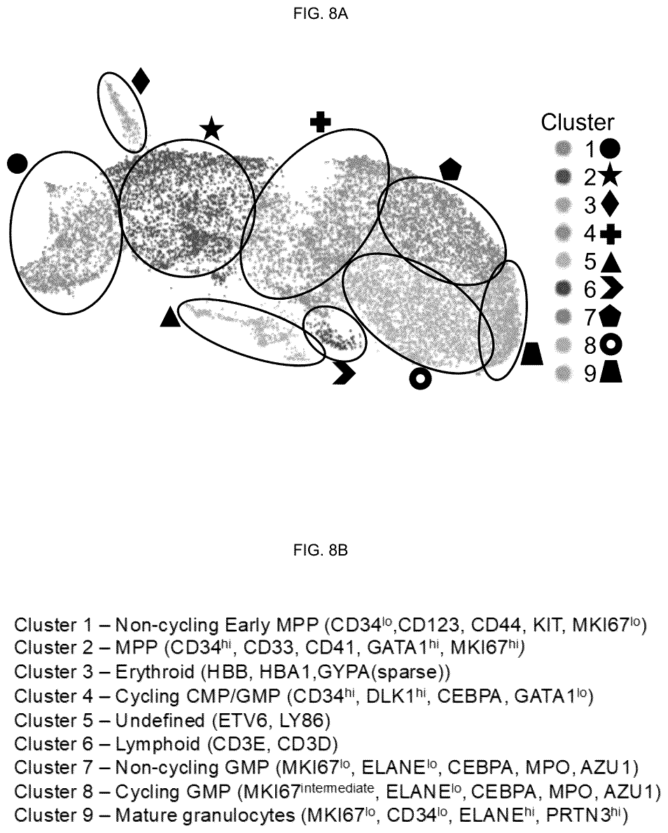

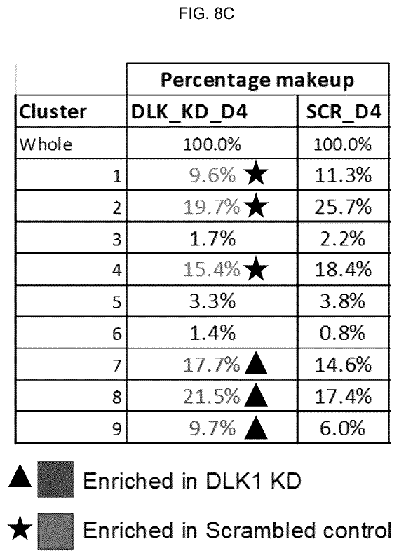

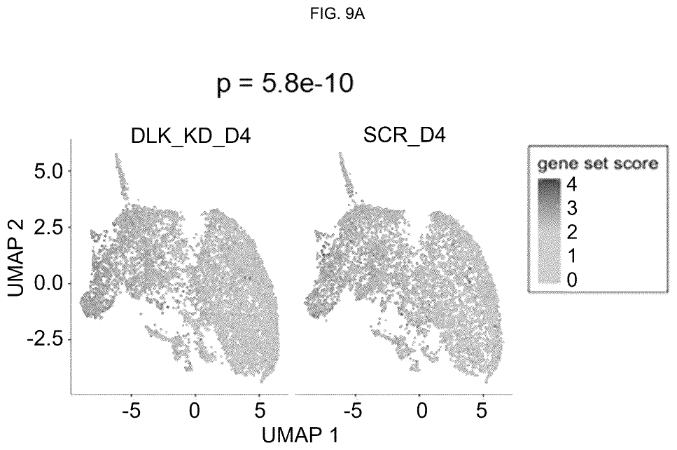

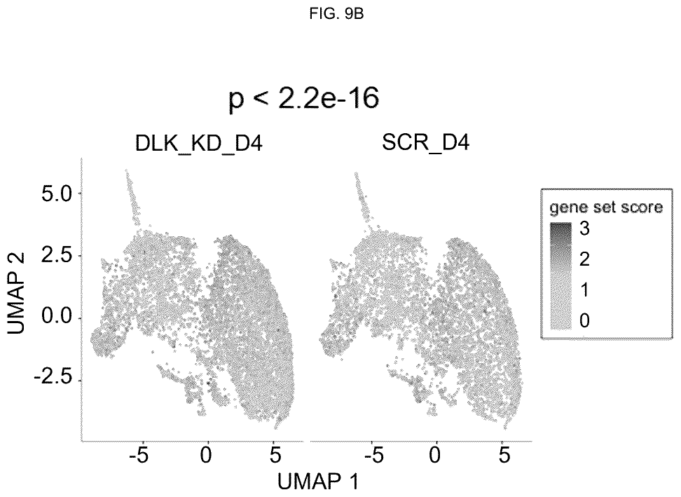

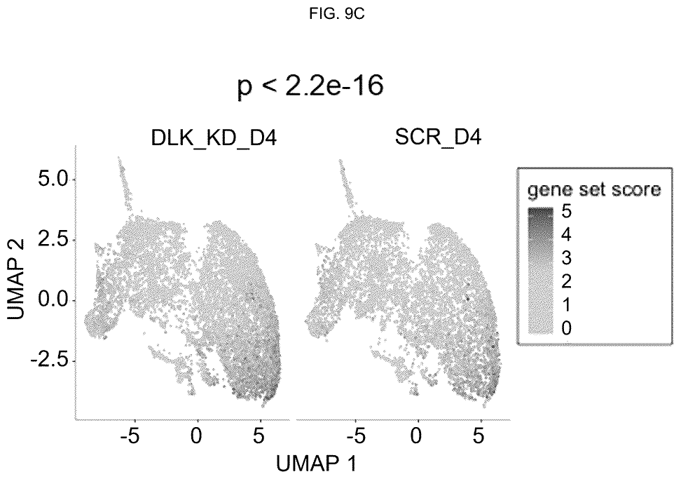

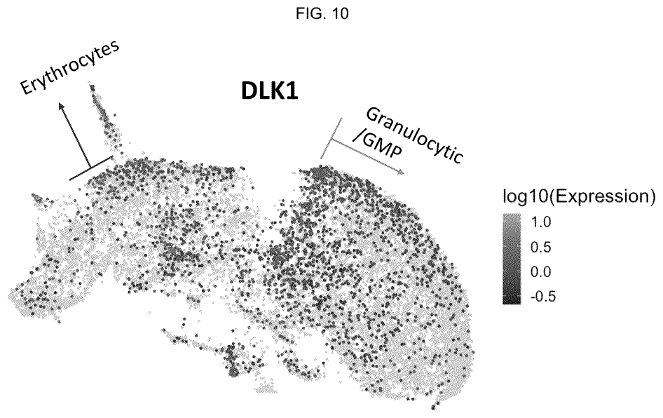

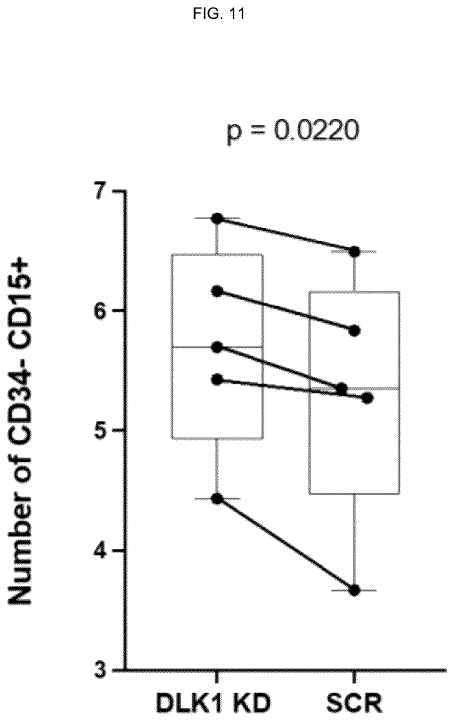

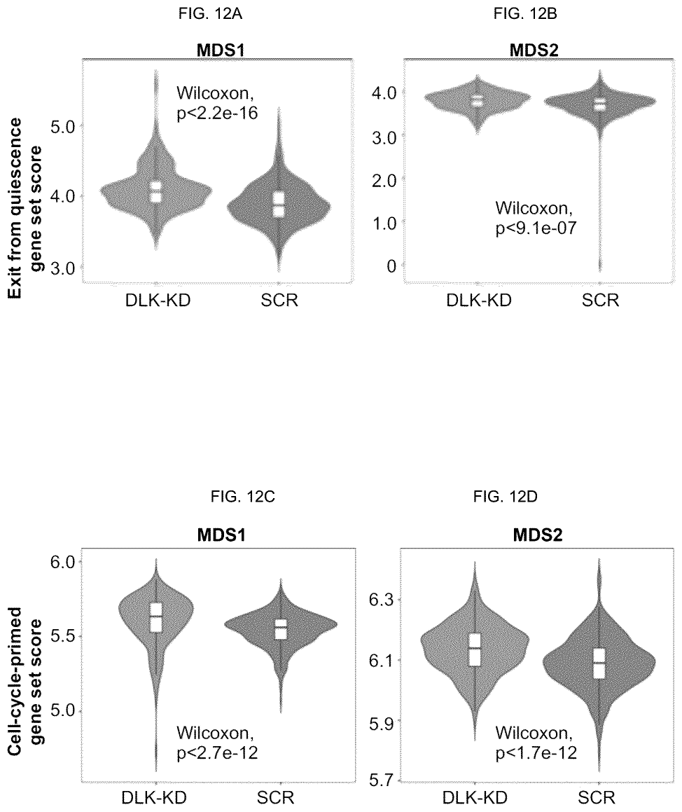

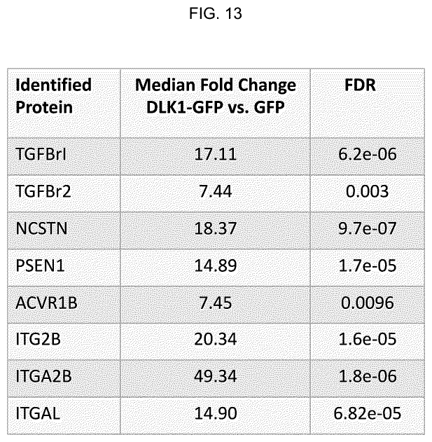

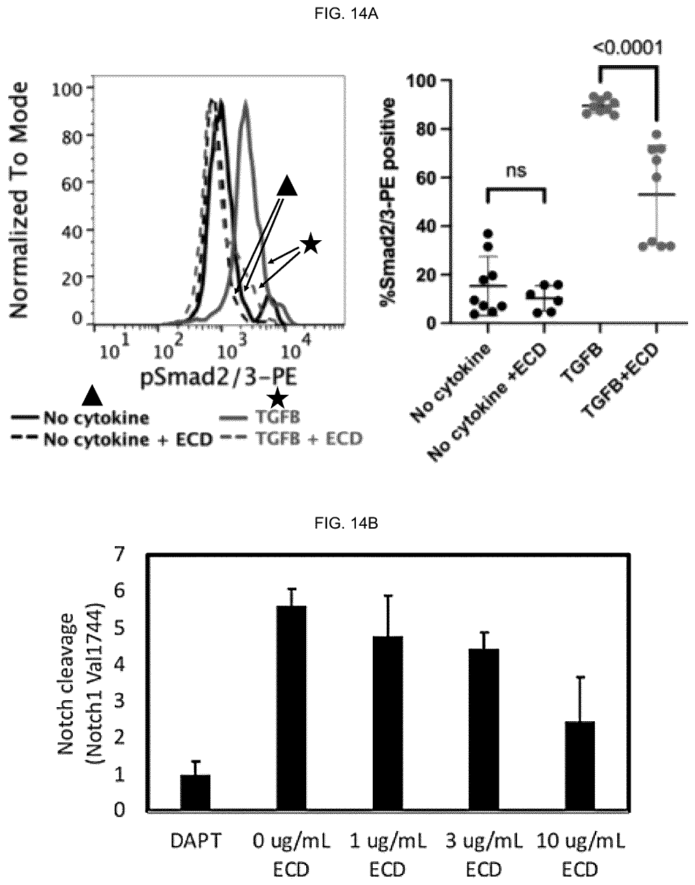

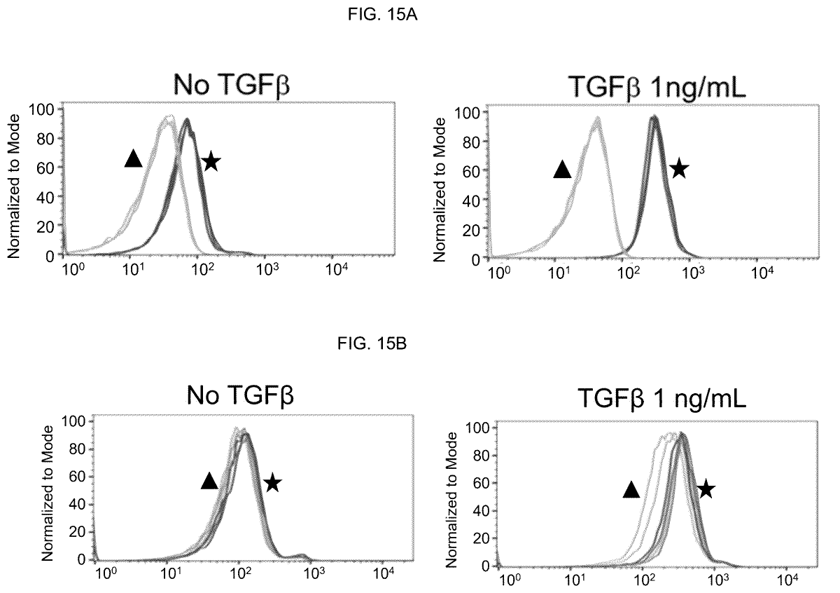

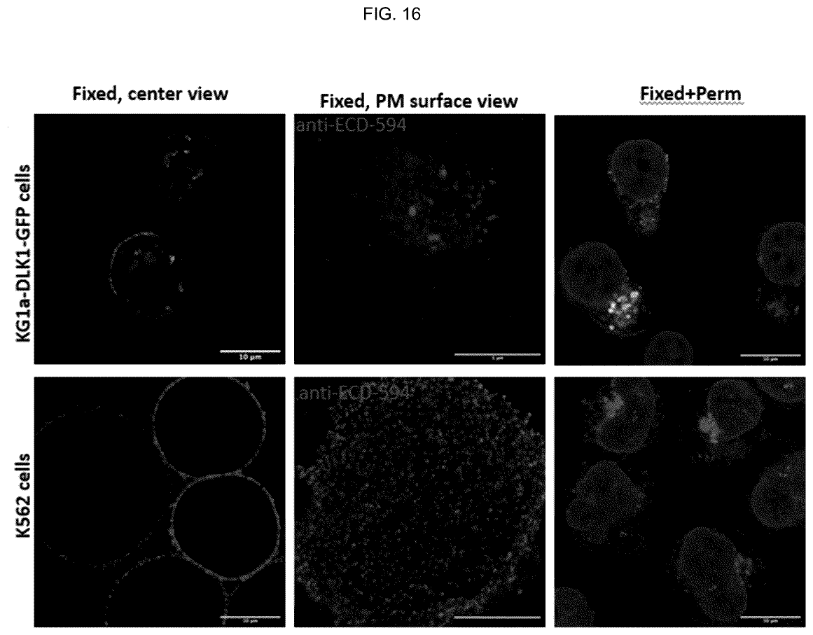

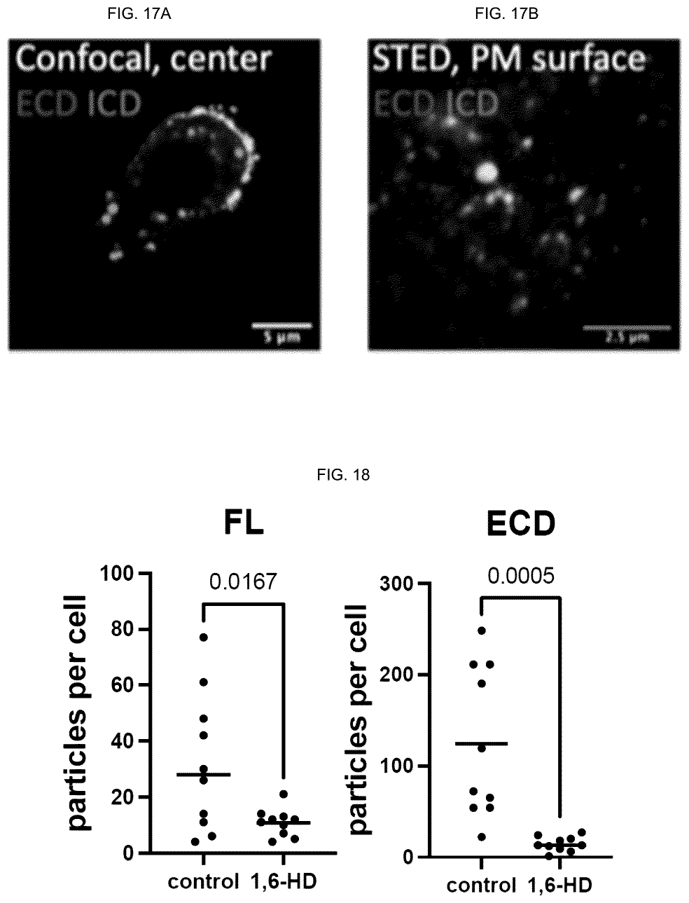

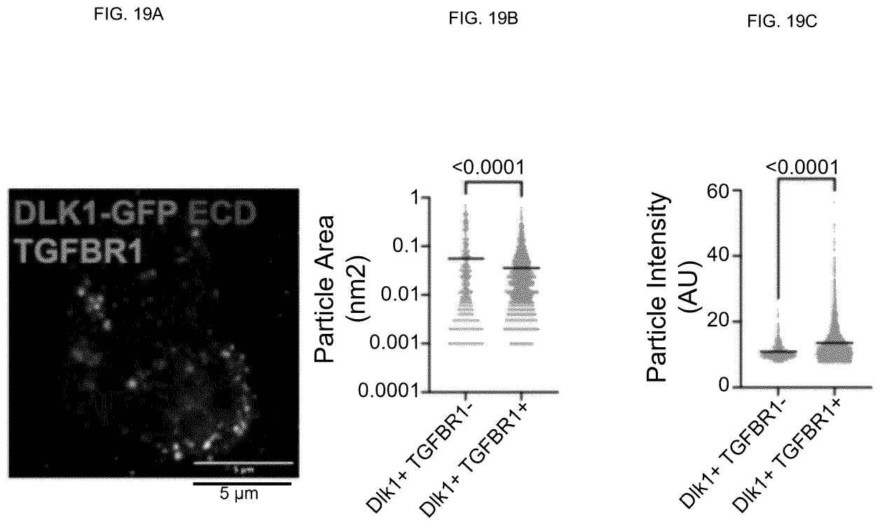

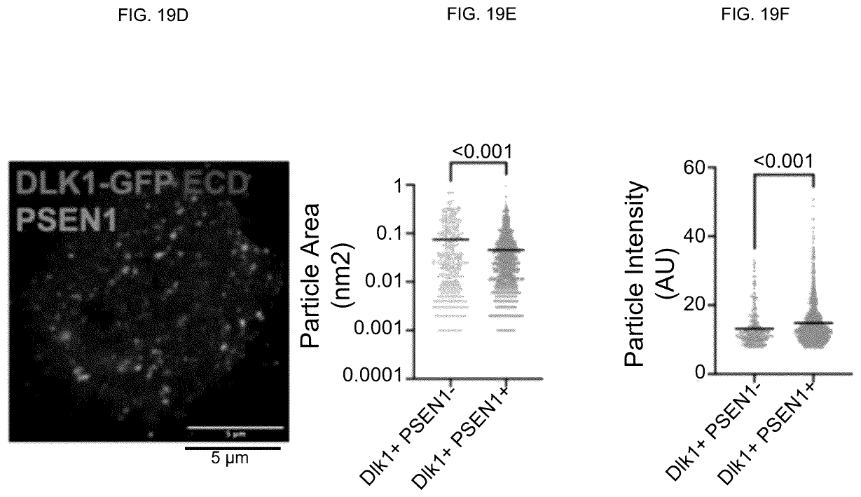

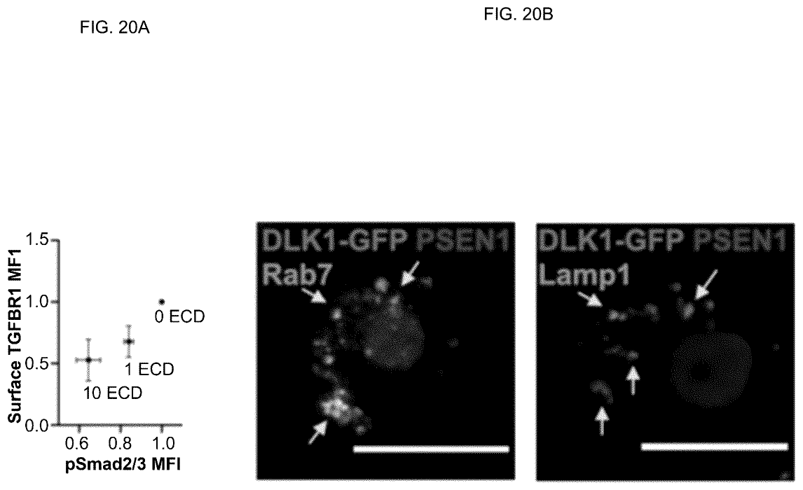

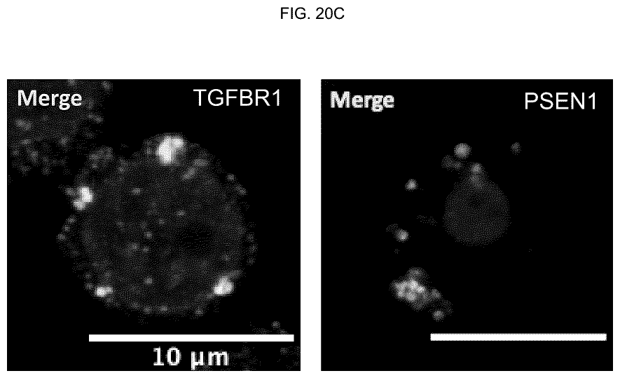

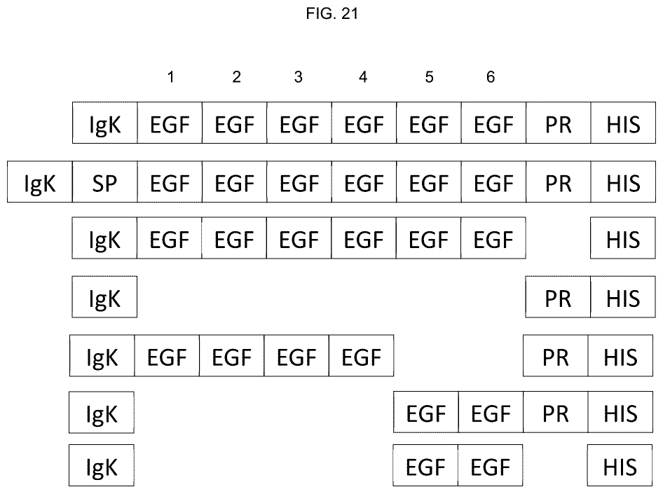

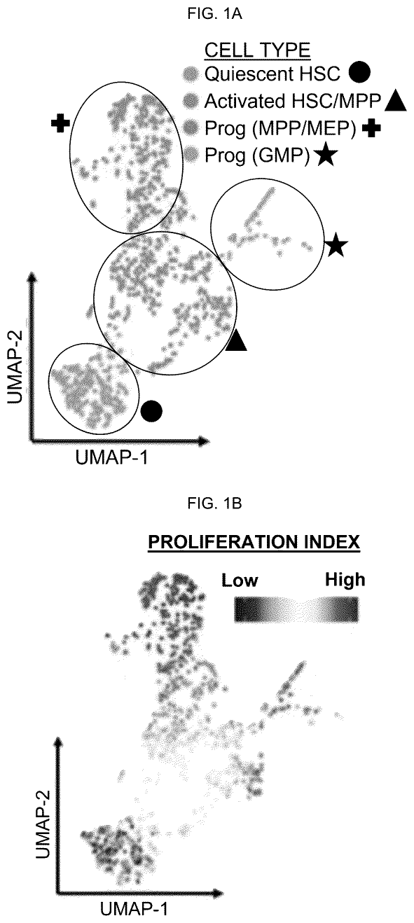

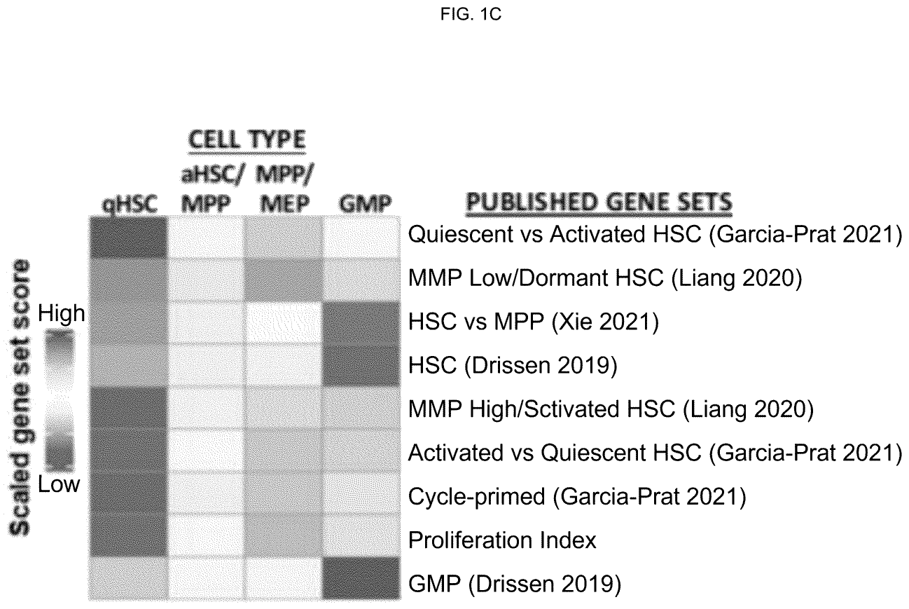

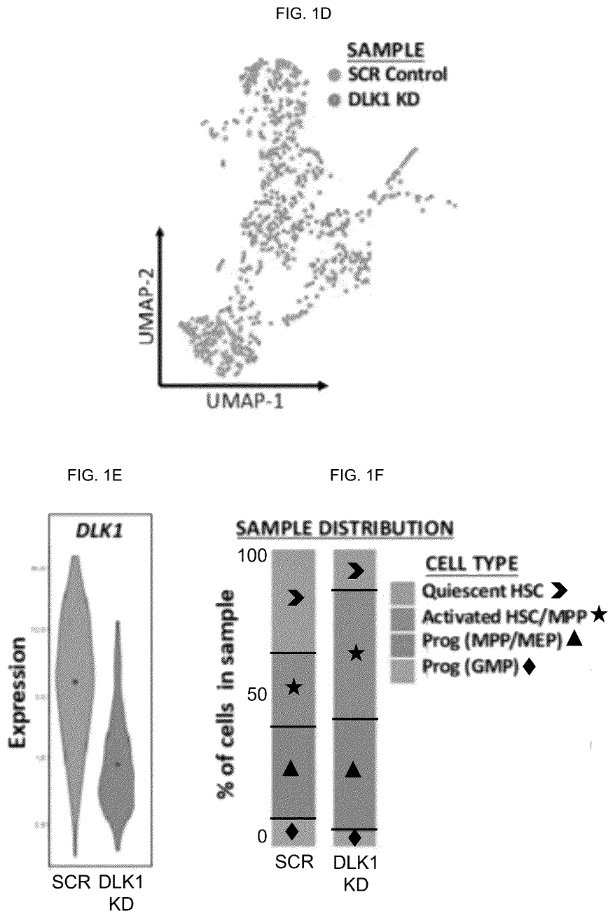

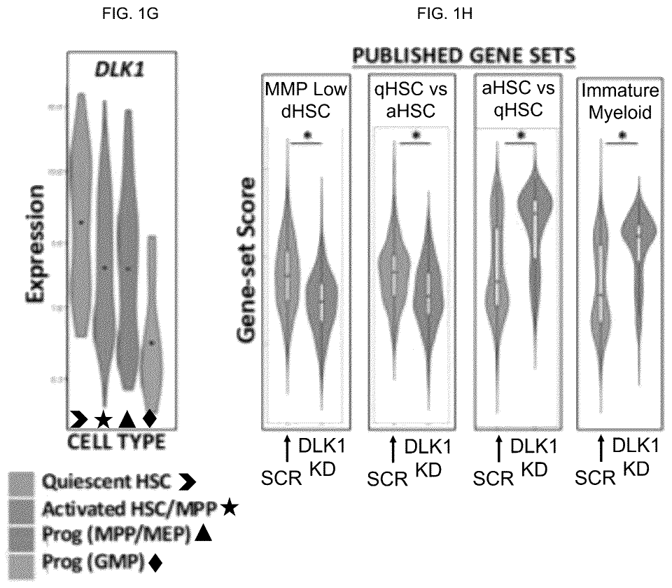

Changes in gene expression that accompany DLK1 knockdown was investigated to gain insight into the molecular pathways involved in DLK1-mediated maintenance of HSC. Using flow cytometry, highly enriched HSC (CD34+Lin-CD38-CD90loCD45RA-EPCR+) were isolated from human CB, a subpopulation known to display higher DLK1 expression than more committed progenitors or differentiated cells [Notta et al., Science, 2011. 333(6039): p. 218-21]. Cells were immediately infected with lentivirus expressing DLK1 shRNA or its scrambled control and cultured for 4 days ex vivo. Cells were harvested and subjected to scRNAseq using the 10× Genomics platform. Dimensionality reduction via Uniform Manifold Approximation and Projection (UMAP, [McInnes & Healy, ArXiv, 2018. abs/1802.03426]) in combination with Louvain clustering [Blondelet et al., Fast unfolding of communities in large networks. Journal of Statistical Mechanics-Theory and Experiment, 2008] and supervised cell type classification using canonical marker genes and published gene sets [Garcia-Pratet al., Cell Stem Cell, 2021. 28(10): p. 1838-1850 e10; Liang et al., Cell Stem Cell, 2020. 26(3): p. 359-376 e7; Drissen et al., Sci Immunol, 2019. 4(35)] identified a quiescent HSC (qHSC) subset, an activated/cycling HSC or multipotent progenitor (aHSC/MPP) subset, and progenitors starting to differentiate along the megakaryocyte/erythroid (MEP) or granulocyte/monocyte (GMP) lineages (FIGS. 1A-1C). Notably, compared to scrambled control cells (SCR), DLK1 knockdown led to a statistically significant decrease in the proportion of qHSC and a corresponding increase in the proportion of aHSC/MPP (Chi-squared p value=1.48e-14). Furthermore, compared to control (SCR), DLK1 knockdown resulted in a significant reduction in gene set scores from published gene signatures associated with metabolically dormant (i.e. low mitochondrial membrane potential (low MMP)) or quiescent HSC state along with a corresponding increase in gene set scores associated with activated/cycling HSC state and early/immature myeloid differentiation [Velten et al., Nat Cell Biol, 2017. 19(4): p. 271-281; Garcia-Prat et al., Cell Stem Cell, 2021. 28(10): p. 1838-1850 e10; Liang et al., 2020. 26(3): p. 359-376 e7] (FIGS. 1D-1H). These data suggest that DLK1 expression inhibits the emergence of HSC from a quiescent state and, upon loss of DLK1, HSC enter the cell cycle to proliferate and differentiate. Taken together, these results have led to the hypothesis that DLK1, via its ECD, maintains the multi-lineage repopulating potential of HSC by preventing their exit from or promoting their return to quiescence.

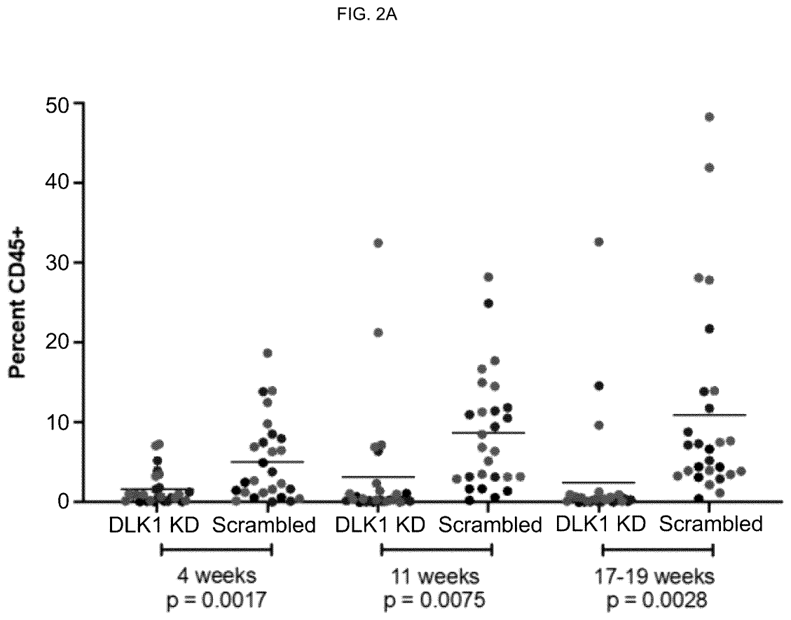

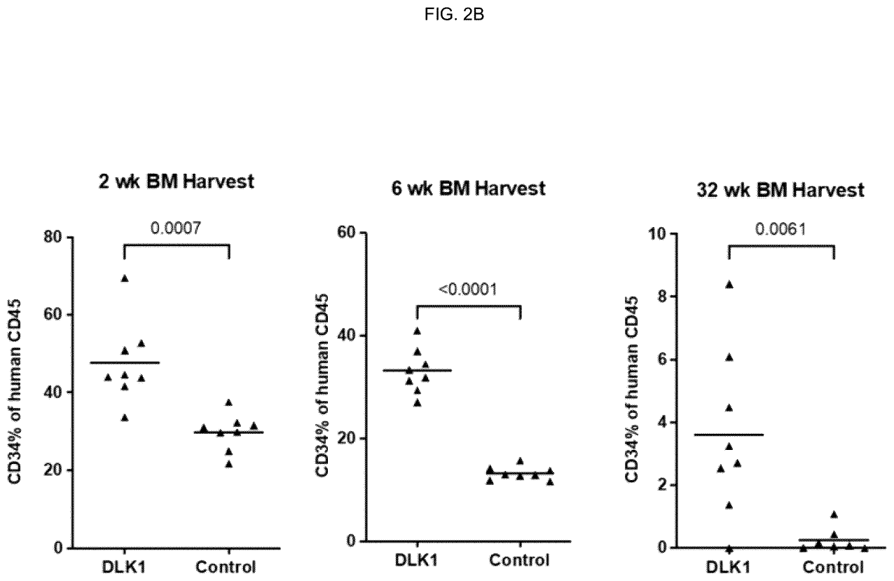

Effect of DLK1 on HSPC: HSPC: DLK1 knockdown reduces in vivo repopulation, whereas DLK1 overexpression delays in vivo CD34+ cell differentiation. To determine whether loss of DLK1 alters HSPC function in vivo, the repopulating ability of CB CD34+ cells following DLK1 knockdown and DLK1 overexpression was assessed. To do so, cryopreserved CB CD34+ cells were thawed and allowed to recover by overnight stimulation in Stemspan plus five growth factors (SS+SGF; SCF, FLT3L, IL-6, TPO, IL-3). Cells were subsequently infected with lentiviruses encoding either 1) DLK1 or scrambled control shRNAs and co-expressing GFP, a marker for viral integration or 2) DLK1-GFP or control GFP. 24 hours post-infection, all cells from each group were transplanted into sub-lethally irradiated, immune-deficient NOD-scid IL2Rgammanull (NSG) mice. For DLK1 knockdown, GFP+ human engraftment in the marrow was determined at 4, 11 and ≥17 weeks post-transplant. DLK1 knockdown significantly reduced both short (4 weeks; p=0.0017) and long term (11 weeks, p=0.0075; 17-19 weeks, p=0.0028) repopulation of hematopoietic cells (FIG. 2A). Furthermore, upon assessment of multi-lineage engraftment, DLK1 knockdown, while reducing the generation of both the myeloid (CD33+) and lymphoid (CD19+) lineages, led to a significant bias towards myeloid differentiation (p-value=0.0372; data not shown). For DLK1 overexpression, GFP+human engraftment was determined for three independent experiments at the time of bone marrow harvest, at 2-, 6- or 32-weeks post-transplant. While not affecting overall human engraftment, DLK1 overexpression led to a statistically significant increase in CD34+ engrafting cells, pointing to delayed differentiation in vivo (FIG. 2B). Taken together, these data suggest that DLK1 regulates HSPC growth and differentiation in vivo.

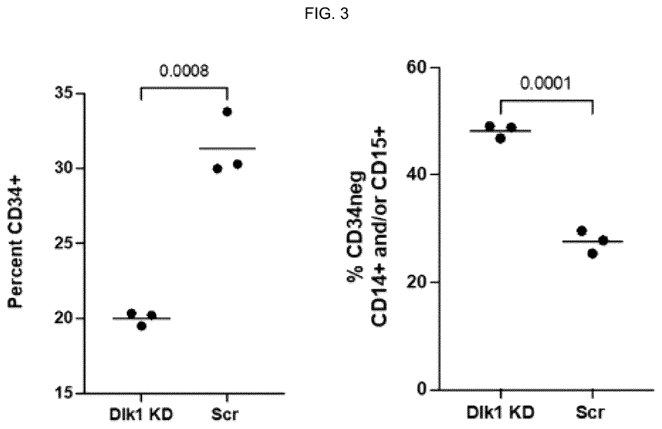

DLK1 knockdown leads to more rapid myeloid differentiation of HSPC during in vitro EC co-culture. To directly test whether DLK1 promotes HSPC maintenance and/or self-renewal by delaying differentiation, CB CD34+ HSPC were transduced with lentivirus encoding either DLK1 or scrambled control shRNAs and co-expressing GFP. Resulting cells were subjected to 14 day coculture with Akt-activated ECs, an approach previously employed to maintain HSC ex vivo [Butler et al., Cell Stem Cell, 2010. 6(3): p. 251-64; Walter et al., Leukemia, 2014. 28(10): p. 1969-77; Le et al., Leukemia, 2021. 35(2): p. 601-605], in the Stemspan+SCF, FLT3L, TPO, all at 50 ng/mL, at which time their differentiation was assessed by flow cytometry. Immuno-phenotype of GFP+ cells was determined by flow cytometry. As shown in FIG. 3, DLK1 knockdown led to a significant decrease in CD34+ percentage (left) as well as an increase in differentiation along the myeloid lineage (right), as characterized by increased CD14 and/or CD15 expression. P-value were determined using a t-test. These findings further suggest that upon reduction of DLK1 expression, HSPC more rapidly differentiate with a bias toward the myeloid lineage.

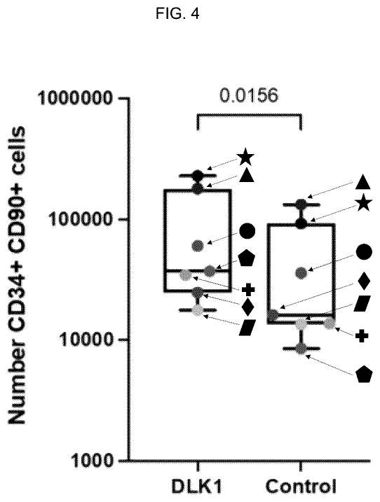

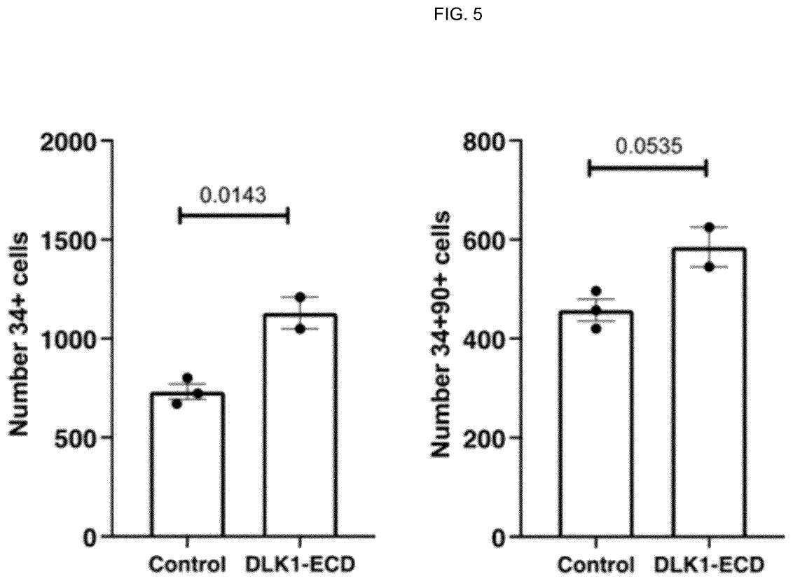

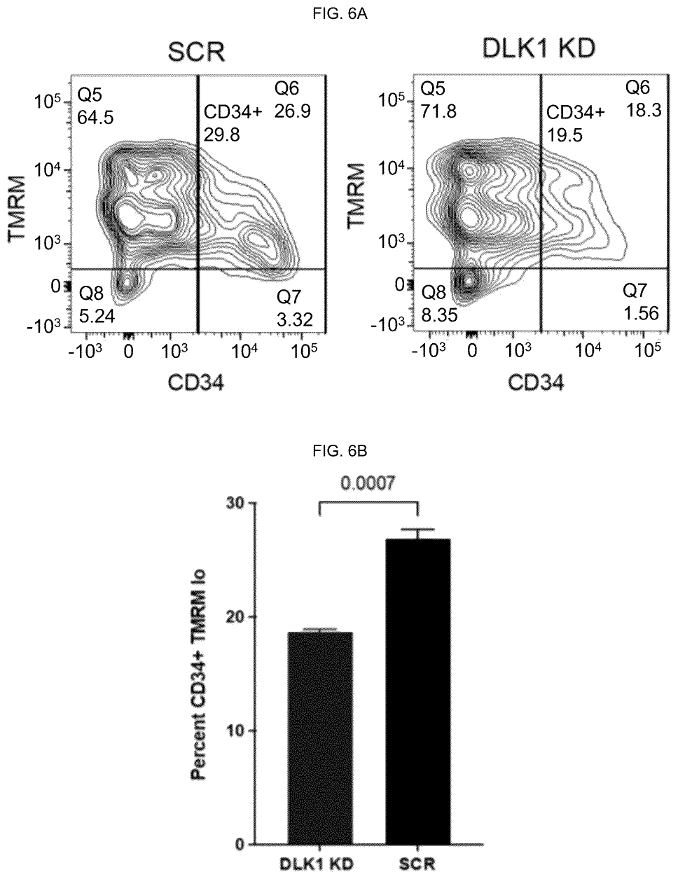

DLK1 overexpression or exposure to exogenous DLK1 ECD leads to an increase in the number of CD34+CD90lo HSPC in vitro. Whether DLK1 overexpression altered the in vivo growth and differentiation of less mature precursors was next assessed. To do so, CB CD34+ HSPC were transduced with lentivirus encoding either DLK1-GFP or Control GFP. Cells were cultured for nine days in the presence of SS+5GF. As shown in FIG. 4, in 7 independent experiments, relative to starting cells, DLK1 overexpression led to a statistically significant increase in GFP+CD34+CD90lo HSPC compared to control (p value=0.0156). These data suggest that DLK1 overexpression is sufficient to enhance the self-renewal and/or induce a delay in the differentiation of the CD34+CD90lo HSPC subset.

As described above, DLK1 can function in both membrane-bound and soluble (ECD) capacities, the latter of which can act in both an autocrine and paracrine manner. Consequently, it was assessed whether exogenously presented ECD was capable of delaying differentiation of highly enriched CB derived HSC during ex vivo culture. To do so, isolated CD34+Lin-CD38-CD90loCD45RA-EPCR+cells were cultured for 9 days in SS+5GF in the presence and absence of ECD. As shown in FIG. 5, compared to control, exogenously presented DLK1 led to an increase in HSPC, with statistically significant increases in CD34+ and CD34+CD90lo HSPC. These data suggest that ECD is sufficient to induce a delay in the differentiation and/or enhance the self-renewal of the CD34+ and CD34+ CD90lo HSPC subsets, implicating the ECD as key to promoting HSC maintenance and/or self-renewal.

To this point, described studies have characterized phenotypic CB-derived HSC as CD34⁺Lin⁻CD38⁻CD90^{lo}CD45RA⁻EPCR⁺. Recent studies have considered the importance of metabolic state to further enrich for the most quiescent subset, characterized by low MMP, as defined by low tetramethylrhodamine ethyl ester (TMRE^{lo}) staining [Liang et al., Cell Stem Cell, 2020. 26(3): p. 359-376 e7; Qiu et al., Blood Adv, 2021. 5(6): p. 1605-1616; Vannini et al., Nat Commun, 2016. 7: p. 13125; Sukumar et al., Cell Metab, 2016. 23(1): p. 63-76]. The effect of DLK1 knockdown on the generation of TMRE^{lo} CD34+ cells was assessed following EC co-culture. DLK1 knockdown led to a 30% loss in TMRM^{lo} CD34+ cells during EC co-culture (FIG. 3), emphasizing the need to focus specifically on the quiescent HSC to test the hypothesis that DLK1 delays differentiation by maintaining/promoting a quiescent stem cell state.

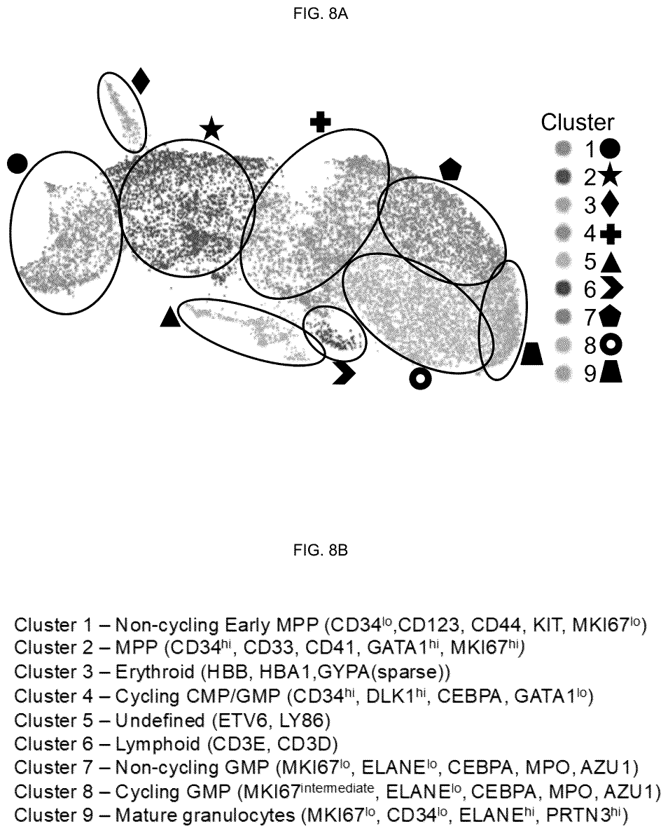

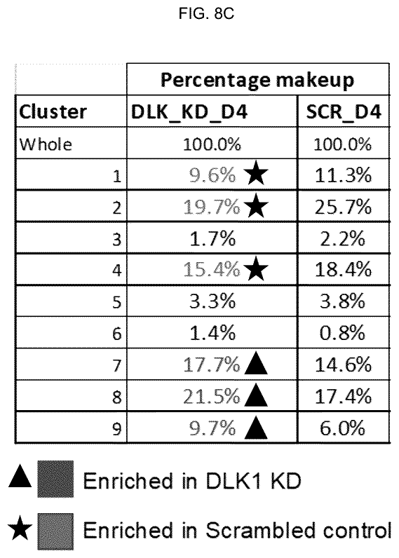

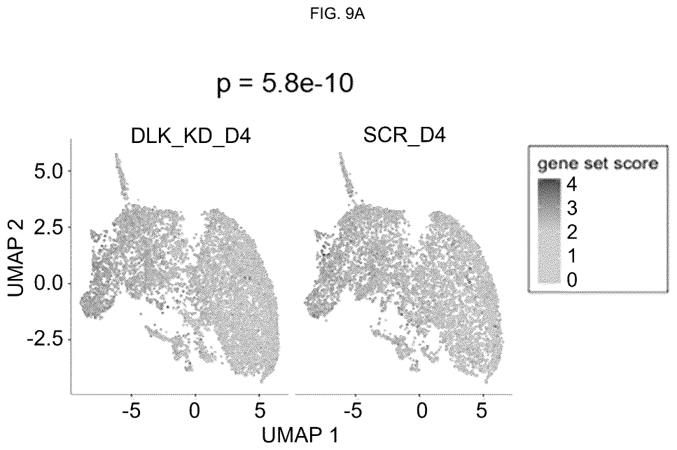

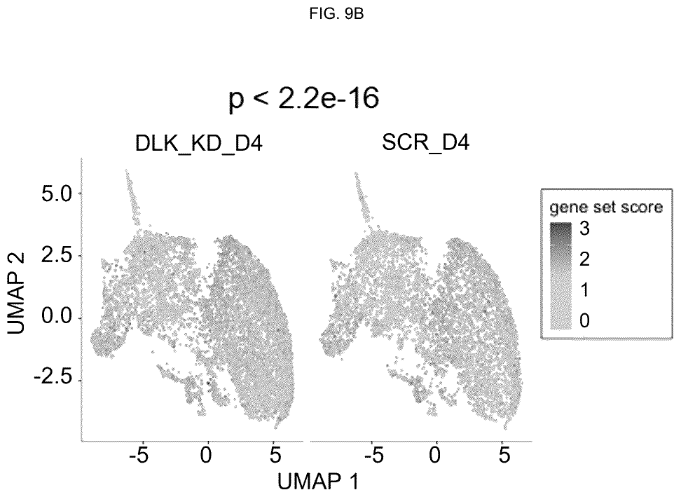

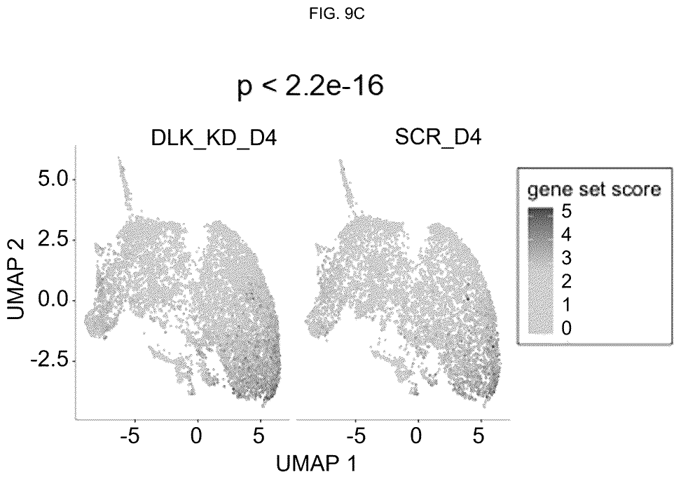

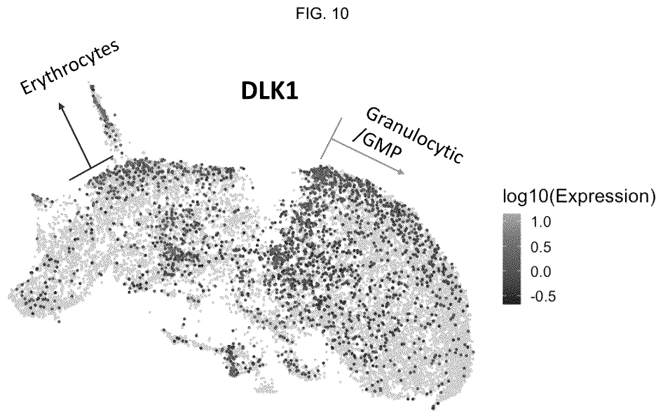

DLK1 reduction enhances the generation of granulocytes at the expense of MDS precursor expansion following short term culture. Dimensionality reduction and clustering on scRNAseq data obtained following induction of DLK1 knockdown and 4 days of short-term in vitro culture identified 9 predominant expression profiles among cells within the combined DLK1 knockdown and scrambled control datasets (FIG. 8A). Clusters were annotated according to the expression of lineage specific markers, as presented in FIG. 8B. Interestingly, DLK1 expression is localized primarily to the multipotent progenitor population (MPP, cluster 2) adjacent to the erythroid cluster as well as along the cusp of cells entering the myeloid lineage (cluster 4) (FIG. 10). Upon DLK1 knockdown, a 65% reduction in DLK1 expression was observed accompanied by 1) a 24% decrease in the proportion of multipotent progenitors (cluster 2), 2) a 22.5% increase in the generation of granulocytic precursors (clusters 7 & 8) and 3) a striking 62% increase in differentiated granulocytes (cluster 9) (FIG. 8C). To further support these observations, aggregate gene expression by gene set score was assessed using previously published HSC/MPP, immature myeloid and granulocyte gene sets (Furlan et al., Blood Adv. 2020; 4(8):1594-605, Saunders et al., Elife. 2019; 8, Roy et al., Cell Rep. 2021; 36(11):109698, Santos et al., Peer J. 2015; 3:e1054; Dignum et al., Cell Rep. 2021; 36(11):109675). As shown in FIG. 9, it was found that DLK1 knockdown led to a significant increase in the immature myeloid and granulocytic gene set scores (p<2.2e-16) as well as an accompanying decrease in HSC/MPP gene set score (p=5.8e-10) compared to Scrambled control cells. Additionally, since this patient was known to harbor chromosomal abnormalities, including monosomy 7 and del 5q, all the cells following short-term culture were able to be identified as being clonal in origin using the algorithm inferCNV (https://github.com/broadinstitute/inferCNV) (data not shown). Taken together, these data show that DLK1 acts on primitive precursors preventing their differentiation towards the myeloid lineage with DLK1 reduction leading to an increase in granulocytic differentiation at the expense of MDS precursor expansion. These data are of clinical relevance as they show the utility of DLK1 inhibition as a way to 1) limit the expansion and possibly decrease the number of blast cells, thereby restraining disease progression, and 2) overcome the neutropenia observed in advanced MDS patients.

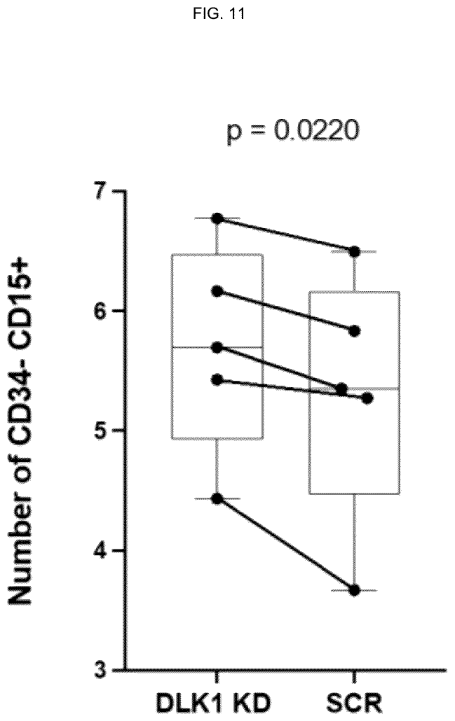

DLK1 reduction enhances the ex vivo generation of granulocytes from MDS precursor cells in longer-term culture. The above studies show that reducing DLK1 expression in MDS precursors enhances differentiation towards the granulocytic lineage following short term in vitro culture. Using five MDS patient samples, these findings were validated using a longer term EC co-culture system, an approach that had previously been employed (Butler et al., Cell Stem Cell. 2010; 6(3):251-64, Walter et al., Leukemia. 2014; 28(10):1969-77, Le et al., Leukemia. 2021; 35(2):601-5) to maintain HSC as well as AML precursors ex vivo. CD34⁺ MDS precursors were isolated and transduced with lentivirus encoding DLK1 shRNA or its scrambled control and co-expressing GFP. Following transduction, cells were immediately plated onto Akt-activated ECs. At 2- and 3-weeks post-transduction, immunophenotype of the GFP⁺ cells within a portion of the culture was analyzed by flow cytometry. FIG. 11 shows that DLK1 knockdown led to an increase in the number of CD34⁻CD15⁺ granulocytes. These data are consistent with the enhanced granulocyte differentiation observed following DLK1 knockdown and short-term culture in the scRNAseq analysis described above. To ensure that these EC-derived precursors retained their clonogenic properties and to test their malignant or normal origin, showing that the enhanced growth of granulocytic precursors and granulocytes were derived, at least in part, from clonal, more primitive malignant precursors with high proliferative potential.

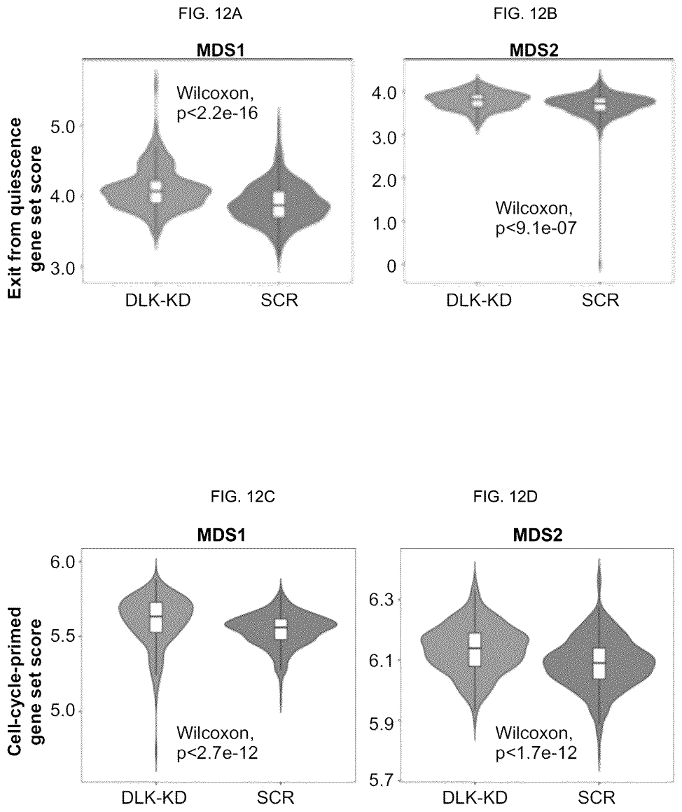

In support of the hypothesis that DLK1 expression maintains or expands the most primitive MDS precursor population by delaying its growth and differentiation, evidence is provided that DLK1 knockdown leads to LSPC exiting quiescence. scRNAseq analysis of the most primitive stem cell cluster, characterized as DLK1-expressing, non-cycling and enriched in stem cell associated genes has been extended. The quiescent state of the most primitive cluster from each individual dataset was further assessed. Two of these three specimens show an increased expression of published gene sets indicative of cells exiting quiescence [Laurenti et al., Cell Stem Cell, 2015. 16(3): p. 302-13] or cells in G0/G1 that exhibit priming for cell cycle in contrast to more deeply quiescent cells [Xie et al., Cell Stem Cell, 2019. 25(5): p. 639-53] (FIG. 12), further suggesting that DLK1 is a key player in the maintenance of LSPC quiescence.

Identification of DLK1 interacting proteins. Despite the identification of DLK1 over twenty years ago, little remains known regarding its mechanism of action. Although multiple protein targets have been shown, their interactions are not supported by the structural properties of DLK1. Based on the notion that DLK1 weakly interacts with its targets, it was assessed whether one could reproducibly detect DLK1 association with its multiple binding partners using a proteomic approach. To do so, a hematopoietic stem cell line (KG1a) expressing the full-length DLK1 isoform with a C-terminal fusion to GFP was generated and used to perform DLK1 immunoprecipitation followed by mass spectrometry (IP-MS). Cells were lysed under conditions that allow solubilization of both membrane-bound and cytoplasmic proteins. DLK1 and its interacting proteins were immunoprecipitated using anti-GFP resin and the immunoprecipitated material subjected to mass spec analysis. 649 proteins were found that were reproducibly immunoprecipitated with DLK1-GFP but not GFP alone (Fold Change>2; FDR<0.01). The web-based gene ontology algorithm Panther (Mi et al., Nucleic Acids Res. 2021; 49(D1):D394-D403) was utilized to focus in on proteins enriched among the cellular component ontology and belonging to subsequent membrane or receptor complex GO terms (GO:0016020 and GO:0043235) that are known to be involved in hematopoiesis. In addition, due to the numerous published reports of DLK1 interacting with the Notch pathway, a biased approach was taken and proteins associated with the Notch signaling pathway were included. A table of the prioritized DLK1 interacting proteins are presented in FIG. 13.

DLK1 acts at least in part by interaction with the TGFb and Notch pathways. Among the list of proteins identified, TGFb receptors (TGFBR1 and TGFBR2) as well as members of the gamma secretase complex (NCSTN and PSEN1) were significantly enriched compared to control-GFP (FIG. 13), showing that DLK1 may affect the TGFb and Notch signaling pathways.

DLK1 Mechanism of action: Despite the identification of numerous proteins consistent with multiple low affinity interactions, several receptor signaling pathways known to regulate HSPC [Traustadottir et al., Cytokine Growth Factor Rev, 2019. 46: p. 17-27] were found. ECD inhibition of two of these pathways, TGFβ and Notch was initially tested. To assess changes in TGFβ signaling, the level of SMAD2/3 phosphorylation (pSMAD2/3) after stimulation with TGFβ ligand was measured (FIG. 14A). To assess changes in Notch signaling, the level of receptor cleavage during co-cultivation of cells with immobilized Notch ligand was measured (FIG. 14B). Importantly, the proteomics study suggested that ECD interacts with Presenilin-1 (PSEN1) and Nicastrin, components of the Gamma Secretase enzyme complex that cleaves several receptors, including Notch. In this experiment, DLK1 did not pulldown Notch and DLK1 is not a substrate of Gamma Secretase as its cleavage pattern was not impacted by Gamma Secretase inhibition with DAPT (data not shown). Supported by data described below, it was hypothesized that DLK1 operates though a central mechanism to inhibit multiple signaling pathways including TGFβ and Notch.

DLK1 plasma membrane organization informs on its function. To observe DLK1 organization, KG1a cells stably expressing DLK1 as a C-terminal GFP fusion protein (DLK1-GFP) were stained with an antibody specific to the N-terminal ECD. Confocal microscopy showed that DLK1 is enriched in the plasma membrane (PM) as well as intracellularly (FIG. 16). This pattern is consistent with endogenously expressed DLK1 in K562 cells.

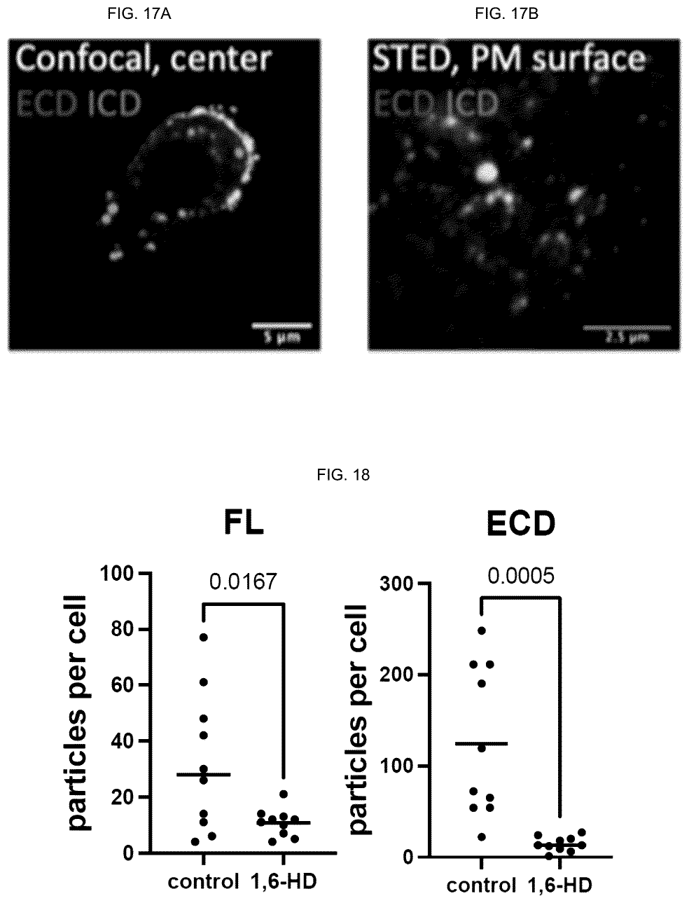

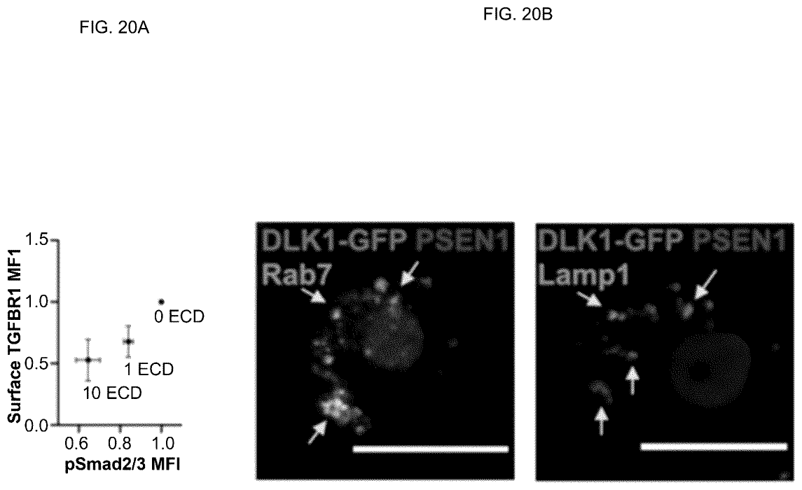

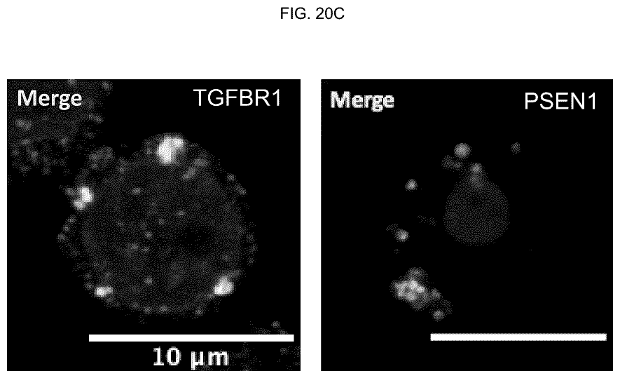

(FIG. 17A). Intracellular DLK1 localizes to late endosomes and lysosomes, determined using immunofluorescence (example in FIG. 20B). Subsequent use of stimulated emission depletion (STED) microscopy revealed that DLK1 in the PM is not uniform but organized into particles that resemble phase separated condensates (FIG. 17B). Using FIJI analysis, the number, size, and intensity of ECD and GFP signals within condensates were determined (data not shown). Consistent with the findings that ECD is sufficient to inhibit TGFβ signaling and Notch cleavage (FIG. 14), cleaved ECD (ECD+/GFP−) represents 75% of the total DLK1 condensates, suggesting ECD is sufficient to phase separate and form condensates.

DLK1 phase separates and co-localizes with receptor components. To address the hypothesis that DLK1 phase separates, whether full-length DLK1 (FL) or ECD condensates are sensitive to 1,6-hexanediol (1,6-HD), which is known to universally disrupt condensates [Liu et al., Genome Biol, 2021. 22(1): p. 230] was investigated. it was found that DLK1 condensates, especially ECD condensates, are sensitive to 1,6-HD, suggesting that DLK1 exhibits phase separation tendencies (FIG. 18).

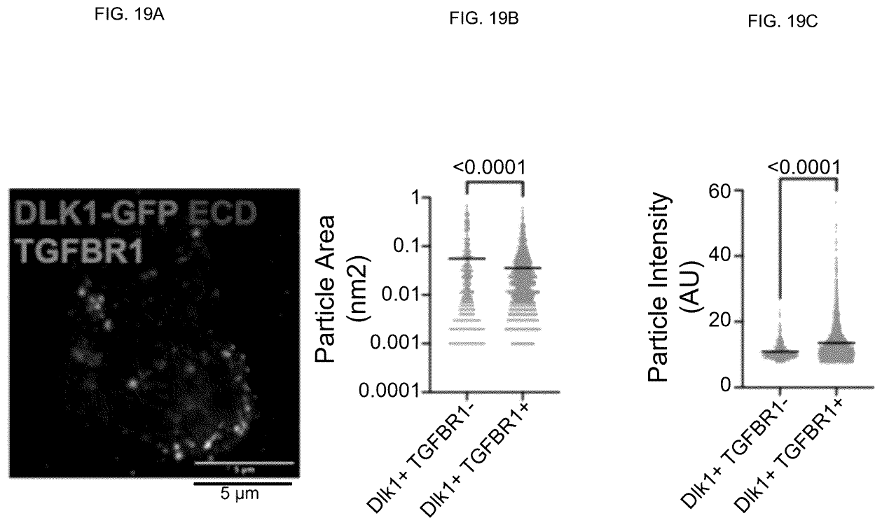

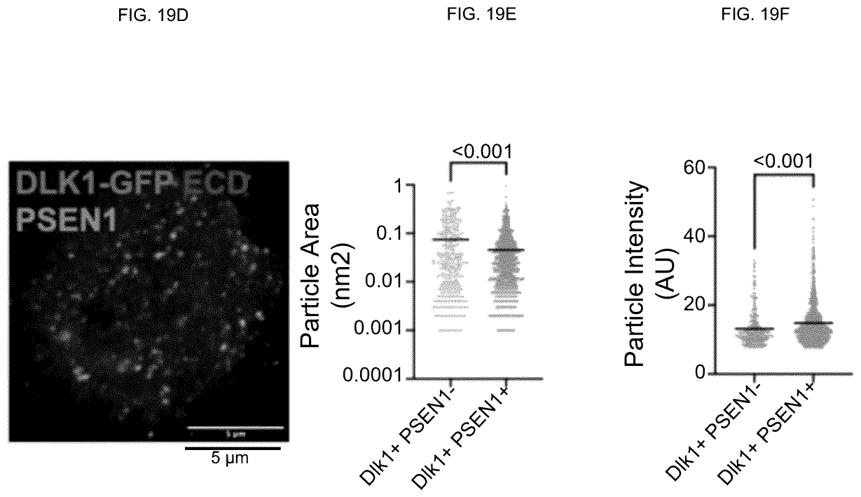

Next, whether DLK1 (FL and ECD) condensates co-localize with TGFBR1 or PSEN1 was assessed. Using STED microscopy to image DLK1 condensates within the PM of KG1a-DLK1-GFP cells (30 nm resolution), co-localization of DLK1 with TGFBR1 was observed (FIG. 19A). Quantitative analysis of 3691 condensates over 10 images reveals that 75% of DLK1 in the PM overlaps with TGFBR1 and 75% of TGFBR1 overlaps with DLK1 data not shown). Similar results were obtained for the colocalization of DLK1 with PSEN1 where analysis of 1864 condensates over 10 images reveals that 80% of DLK1 in the PM overlaps with PSEN1 and 75% of PSEN1 overlaps with DLK1 (data not shown). The overlap of DLK1 with TGFBR1 (75%) and PSEN1 (80%) suggests that TGFBR1 and PSEN1 are likely in the same DLK1 condensates.

Next, it was tested whether DLK1 differed among DLK1+Receptor+and DLK1+Receptor-condensates, with the use of Receptor representing both TGFBR1 and/or the receptor component PSEN1, by assessing condensate size and DLK1 intensity. Despite having a smaller area (FIGS. 19B, 19E), ECD intensity was significantly higher in condensates that overlap with TGFBR1 or PSEN1 suggesting that DLK1 concentration increases in receptor positive condensates, consistent with a phase separation mechanism (FIGS. 19C, 19F, respectively). Given the expected phase separation tendencies of DLK1, especially of ECD, it is hypothesized that the difference in DLK1intensity represents ECD oligomerization and scaffolding with receptor components.

The effect of the ECD on receptor dynamics was further investigated and that surface levels of TGFBR1 decrease in a dose-dependent manner after ECD addition was observed, in part explaining the ECD-dependent decrease in pSmad2/3 levels (FIG. 20A). Notably, this effect did not happen immediately (30 minutes) after ECD addition but required overnight incubation (data not shown), suggesting that DLK1 might affect receptor recycling and/or turnover, a notion further supported by the observation that DLK1 co-localizes with TGFBR1 and PSEN1 in late endosomes and lysosomes (data for PSEN1 shown in FIG. 20B). Overall, these data suggest that ECD phase separation with TGFBR1 and PSEN1 in the PM impedes signaling by inhibiting receptor or receptor component dynamics.

The studies described here show a conceptually innovative mechanism whereby DLK1 preserves HSPC quiescence by undergoing phase separation to form condensates that inhibit the response of cell surface receptors to multiple environmental signals that induce growth and differentiation. Such findings represent a significant advance in understanding stem cell biology and show a novel biological consequence to phase separation. Uses include the expansion of true repopulating HSC for transplantation and/or enhancing the effectiveness of HSC-based gene therapy as well as the prevention of HSC aging and its consequences, including the occurrence of CHIP. Since DLK1 is mis-expressed in many aggressive, treatment resistant cancers, including Myeloid Dysplastic Syndrome as well as common solid tumors [Grassi & Pietras, J Histochem Cytochem, 2022. 70(1): p. 17-28; Pittaway et al., Endocr Relat Cancer, 2021. 28(12): p. R271-R287], the findings described herein are directly applicable to the development of DLK1 inhibition strategies to reduce disease progression, including rendering cancer stem cells susceptible to conventional chemotherapy.

TGFβ: 24 hours post-transduction, cells will be harvested to assess changes in pSMAD2 induced by autocrine expressed TGFβ. A flow cytometric approach (Schulz et al., Curr Protoc Immunol. 2007; Chapter 8:Unit 8 17) will be employed using an antibody (clone O72-670; BD Biosciences) that recognizes both human pSMAD2 and pSMAD3. Flow analysis will assess GFP expressing DLK1 knockdown or Scrambled control cells for pSMAD2/3 compared to isotype control antibody. FIG. 15A shows the feasibility of the assay in detecting pSMAD2/3 in response to both autocrine expressed (left panel) as well as exogenously added TGFβ ligand (right panel) using 25,000 wild type KG1a cells, a number obtainable in the MDS studies. Furthermore, FIG. 15B shows the feasibility of the assay in detecting pSMAD2/3 in response to both autocrine expressed (left panel) as well as exogenously added TGFβ ligand (right panel) in KG1a cells transduced to expressed DLK1-GFP or GFP control. A DLK1-GFP induced reduction in pSMAD2/3 was found in response to exogenously added TGFβ ligand (right panel), with a more subtle reduction in response to autocrine expressed TGFβ. The data show that both endogenously expressed DLK1 or soluble ECD are capable of inhibiting pSMAD2 in response to TGFβ ligand. As a control in the MDS studies, pSMAD2/3 in the presence of blocking anti-TGFβ antibodies will be assessed to confirm that the baseline pSMAD2/3 observed is induced by autocrine TGFβ (as shown for the KG1a cell experiments (data not shown)).

Notch. In contrast to the TGFb experiments, immediately following transduction with lentivirus encoding DLK1 shRNA or its scrambled control and co-expressing GFP, MDS precursors will be transferred to culture wells coated with immobilized engineered Delta1 ligand (Delaney et al., Nat Med. 2010; 16(2):232-6, Varnum-Finney et al., J Cell Sci. 2000; 113 Pt 23:4313-8), which leads to Notch receptor cleavage by theg-secretase complex and subsequent target gene activation. Cells will be harvested 24 hours post-transduction and used as input for bulk RNAseq analysis to simultaneously assess whether DLK1 knockdown leads to a reduction in known Notch target gene expression, including HES1, HEY1, DTX1, and NRARP, among GFP⁺ cells. The extent of the DLK1 effect will be compared to the levels of Notch activation observed in MDS precursors following treatment with Notch signaling antagonists, including-secretase inhibitors.

Closing Paragraphs. Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1. A modified DLK1 extracellular domain (ECD), wherein the modification is a substitution or deletion of Arg160; a substitution or deletion of Gly199; a substitution or deletion of Arg267; a substitution or deletion of Val272; a substitution or deletion of Arg160 and Gly199; a substitution or deletion of Arg160 and Arg267; a substitution or deletion of Arg160 and Val272; a substitution or deletion of Gly199 and Arg267; a substitution or deletion of Gly199 and Val272; a substitution or deletion of Arg267 and Val272; a substitution or deletion of Glu377; a substitution or deletion of Ala378; a substitution or deletion of Gly379; a substitution or deletion of Asp380; a substitution or deletion of Glu381; a substitution or deletion of Glu382; a substitution or deletion of lle383; a substitution or deletion of residues 377-383; a substitution or deletion of 2 of residues 377-383; a substitution or deletion of 3 of residues 377-383; a substitution or deletion of 4 of residues 377-383; a substitution or deletion of 5 of residues 377-383; a substitution or deletion of Ala171; a substitution or deletion of Ser173; a substitution or deletion of Cys174; a substitution or deletion of Thr186; a substitution or deletion of Asp187; a substitution or deletion of two of residues Ala171, Ser173, Cys174, Thr186, and Asp187; a substitution or deletion of three of residues Ala171, Ser173, Cys174, Thr186, and Asp187; a substitution or deletion of four of residues Ala171, Ser173, Cys174, Thr186, and Asp187; a substitution or deletion of Ala171, Ser173, Cys174, Thr186, and Asp187; a substitution, deletion, insertion, or modification of Cys174; a substitution, deletion, insertion, or modification of Cys179; a substitution, deletion, insertion, or modification of Cys185; a substitution, deletion, insertion, or modification of Cys194; a substitution, deletion, insertion, or modification of Cys196; a substitution, deletion, insertion, or modification of Cys205; a substitution, deletion, insertion, or modification of Cys212; a substitution, deletion, insertion, or modification of Cys217; a substitution, deletion, insertion, or modification of Cys223; a substitution, deletion, insertion, or modification of Cys233; a substitution, deletion, insertion, or modification of Cys235; or a substitution, deletion, insertion, or modification of Cys244.

2-32. (canceled)

33. The modified DLK1 ECD of claim 1, having a modified signal peptide comprising: a substitution or deletion of 1 residue of the signal peptide, 2 residues of the signal peptide, 3 residues of the signal peptide, 4 residues of the signal peptide, 5 residues of the signal peptide, 6 residues of the signal peptide, 7 residues of the signal peptide, 8 residues of the signal peptide, 9 residues of the signal peptide, 10 residues of the signal peptide, 11 residues of the signal peptide, 12 residues of the signal peptide, 13 residues of the signal peptide, 14 residues of the signal peptide, 15 residues of the signal peptide, 16 residues of the signal peptide, 17 residues of the signal peptide, 18 residues of the signal peptide, 19 residues of the signal peptide, 20 residues of the signal peptide, 21 residues of the signal peptide, 22 residues of the signal peptide, or 23 residues of the signal peptide.

34-67. (canceled)

68. The modified DLK1 ECD of claim 1 as part of a full length DLK1 protein.

69. A single chain protein comprising a modified DLK1 ECD of claim 1.

70. The single chain protein of claim 69, further comprising a signal peptide.

71. The single chain protein of claim 70, wherein the signal peptide comprises an IgK signal peptide or a DLK1 signal peptide.

72. (canceled)

73. The single chain protein of claim 69, wherein the modified DLK1 ECD comprises at least two EGF domains.

74. The single chain protein of claim 69, wherein the modified DLK1 ECD comprises 3, 4, 5, or 6 EGF domains.

75. The single chain protein of claim 69, wherein the modified DLK1 ECD comprises EGFS and EGF6.

76. The single chain protein of claim 69, wherein the modified DLK1 ECD comprises EGF1, EGF2, EGF3, and EGF4.

77. The single chain protein of claim 69, wherein the modified DLK1 ECD comprises EGF1, EGF2, EGF3, EGF4, EGF5, and EGF6.

78. The single chain protein of claim 69, further comprising an ADAM17/TNFα converting enzyme (TACE)-dependent proteolytic cleavage site.

79. The single chain protein of claim 69, further comprising a polyhistidine tag.

80. A nucleotide sequence encoding the modified DLK1 ECD of claim 1.

81. The nucleotide sequence of claim 80, wherein the nucleic acid is DNA or RNA.

82. The nucleotide sequence of claim 81, wherein the RNA is messenger RNA (mRNA) or in vitro synthesized RNA.

83-87. canceled

88. A method of promoting hematopoietic stem cell differentiation towards a myeloid lineage comprising administering a therapeutically effective amount of a DLK1 inhibitor disclosed herein to a population of hematopoietic stem cells, thereby promoting hematopoietic stem cell differentiation towards a myeloid lineage.

89. The method of claim 88, wherein the promoting the hematopoietic stem cell differentiation towards a myeloid lineage reduces the expression of stemness markers within the population of hematopoietic stem cells.

90. The method of claim 88, wherein the promoting the hematopoietic stem cell differentiation towards a myeloid lineage drives HSC from a transcriptionally quiescent state to a more differentiated cell cycling state within the population of hematopoietic stem cells.

91. The method of claim 88, wherein the promoting the hematopoietic stem cell differentiation towards a myeloid lineage decreases a proportion of multipotent progenitors within the population of hematopoietic stem cells and increases the proportion of CD34+CD15+ granulocyte precursors and/or increases the proportion of CD34−CD15+ granulocytes within the population of hematopoietic stem cells.

92-118. (canceled)
